(19) United States
(12) Patent Application Publication
(10) Pub. No.: US 2010/0227844 A1
(43) Pub. Date: Sep. 9, 2010

(54) CANNABINOID-1 RECEPTOR MODULATORS USEFUL FOR THE TREATMENT OF ALZHEIMER'S DISEASE

(76) Inventors: Tung M. Fong, Somerset, NJ (US); Leonardus H.T. Van Der Ploeg, Newton, MA (US)

Correspondence Address: MERCK, P O BOX 2000, RAHWAY, NJ, 07065-0907, US

(21) Appl. No.: 12/682,846

(22) PCT Filed: Oct. 20, 2008
(86) PCT No.: PCT/US08/11942

§ 371 Date: Apr. 13, 2010

Related U.S. Application Data
(60) Provisional application No. 60/999,993, filed on Oct. 23, 2007.

(51) Int. Cl. A61K 31/165; A61P 25/28; A61K 31/4412; A61K 31/44; A61K 31/444; A61K 31/513; A61K 31/437; A61K 31/4439; A61K 31/55; A61K 31/397

(52) U.S. Cl. 514/210.01; 514/622; 514/346; 514/357; 514/335; 514/274; 514/269; 514/300; 514/339; 514/215

(57) ABSTRACT

The present invention relates to methods of treating Alzheimer's Disease and Alzheimer's Disease related disorders comprising administration of a compound of structural formula: (I), or a pharmaceutically acceptable salt thereof, as a monotherapy or in combination with an anti-Alzheimer's Disease agent. The present invention further provides for pharmaceutical compositions, medicaments, and kits useful in carrying out these methods.

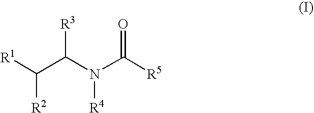

### BACKGROUND OF THE INVENTION

Alzheimer's disease is a neurodegenerative disease characterized by substantial cognitive deficits and functional impairment and progressive loss of memory and general cognitive function. It is the leading cause of dementia in the elderly, affecting more than 4 million people in the US alone. Alzheimer's disease is pathologically characterized by the deposition of extracellular proteinaceous plaques in the cortical and associative brain regions of sufferers. These plaques mainly comprise fibrillar aggregates of β-amyloid peptide (Aβ) (Giulian, American Journal of Human Genetics 65, 13-18 (1999); Wyss-Coray and Mucke, Neuron 35, 419-432 (2002). It is currently thought that aberrant processing of Amyloid Precursor Protein (APP) leads to formation of the Beta Amyloid Peptide Aβ deposits which, in conjunction with other factors, stresses nearby neurons, resulting in tau hyperphosphorylation and induction neurofibrillary tangles (Maccioni et al, Archives of Medicine Research 32, 367-381 (2001). After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD. Reducing the burden of Aβ in the brain has therefore been proposed as a strategy for treatment of Alzheimer's Disease, and various interventions in the plaque-forming process have been proposed as therapeutic treatments (Hardy and Selkoe, Science, 297 (2002), 353-6).

Recent reports have implicated the endocannabinoid system in inflammatory processes in the brain and, specifically, in Alzheimer's Disease. It has been recently reported that CB-1 receptor blockade by SR 141716, a antagonist/inverse agonist, improves the memory deficit induced by Beta Amyloid Peptide Aβ-administration in mice (Mazzola et al. European Journal of Pharmacology 477, 219-225 (2005). It has also been shown that treatment with rimonabant, a CB1R inverse agonist, improves learning and memory performance of naïve as well as scopolamine or Aβ-treated rodent models (Terranova et al., Psychopharmacology, 126:165-172 (1996).

The cannabinoid-1 receptor (CB1R) plays an important role in mediating cognition in both rodents and humans. CB1R knockout mice show enhanced long-term potentiation in a molecular model of memory storage, and improved recognition memory, and show improvements in several memory tasks, including Morris water maze and tone-shock association (Bohme et al., Neuroscience 95: (2000); Varvel, Lichtman, J Pharmacology & Experimental Therapeutics, Vol. 301, No. 3, 915-924 (2002); Reibaud et al., European Journal of Pharmacology, 379; R1-R2, 1999). Cannabinoid-1 (CB-1) receptor agonists are also known to cause short-term memory impairment in humans (Hollister, Pharmacol. Rev. 38:1-20, 1986).

In humans, CB1R agonist treatment, such as treatment with cannabinoids, impairs performance on tests of attention, episodic memory, short-term memory and executive functions (Harold Kalant, Progress Neuro-Psychopharm & Biol Psychiatry 28 (2004) 849-863; De Souza et al., 2005, Biol Psychiat. 57:594). The CB-1 receptors appear to exert their effects on cognition, in part, through presynaptic regulation of the release of several neurotransmitters, including acetylcholine, glutamate, and monoamines. It is known that CB-1 agonists generally inhibit neural transmission. CB-1 receptor inverse agonists may be useful to increase neural transmission, including central acetylcholine (Ach) transmission/release, which is known to be deficient in Alzheimer's Disease. Studies have shown that Rimonabant administered in combination with the acetylcholine esterase Donepezil produces additive effects on cognition scores in a rodent model of episodic memory (Wise et al., Neuropsychopharmacology, 32, 1805-1812 (2007)). These data suggest that CB1R inverse agonists or antagonists may improve cognition in humans and can be used to treat Alzheimer's Disease, memory loss and other memory impairments.

Furthermore, the treatment of Alzheimer's Disease with a combination of a cannabinoid-1 receptor antagonist/inverse agonist and an existing anti-Alzheimer's Disease agent may provide superior efficacy compared to treatment with either agent alone. In particular, treatment of Alzheimer's Disease with a combination of a cannabinoid-1 receptor antagonist/inverse agonist, which increases acetylcholinesterase transmission, and an anti-Alzheimer's Disease agent that works by a different mechanism, such as an acetylcholinesterase (AChE) inhibitor which increases the half life of acetylcholinesterase, may result in synergistic relief.

Approved treatments for Alzheimer's Disease include acetyl cholinesterase inhibitors, such as Donepezil Hydrochloride (Aricept®, and Tacrine Hydrochloride (COGNEX®; and glutamatergic NMDA receptor antagonists such as Memantine (Axura®, Akatinol®, Namenda®, and Ebixa®). These agents produce significant peripheral and gastrointestinal side effects, which may limit their dose, and both are associated with limited efficacy.

It is an object of the present invention to identify compounds useful for the treatment of Alzheimer's Disease. It is another object of the invention to identify methods of treating Alzheimer's Disease and Alzheimer's Disease related disorders. It is yet another object of the invention to identify methods of preventing Alzheimer's Disease and Alzheimer's Disease related disorders. It is a further object of the present invention to provide pharmaceutical compositions comprising a cannabinoid-1 receptor antagonist/inverse agonist and an anti-Alzheimer's Disease agent. It is yet a further object of the present invention to provide a method of manufacture of a medicament useful in the treatment of Alzheimer's Disease and Alzheimer's Disease related disorders.

### SUMMARY OF THE INVENTION

The present invention relates to methods of treating or preventing Alzheimer's Disease and Alzheimer's Disease related disorders comprising administering a therapeutically effective amount of a compound of Formula I

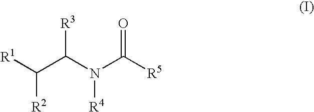

or pharmaceutically acceptable salts thereof. The compounds of formula I are antagonists and/or inverse agonists of the Cannabinoid-1 (CB-1) receptor and are useful in the treatment or prevention of Alzheimer's Disease and Alzheimer's Disease related disorders, including but not limited to, dementia, age related cognitive decline, and mild cognitive impairment.

The present invention is also concerned with treatment of these conditions, and the use of the compositions of the present invention for manufacture of a medicament useful for treating these conditions.

The invention is also concerned with pharmaceutical compositions comprising a cannabinoid-1 receptor antagonist/inverse agonist and an anti Alzheimer's Disease agent, as active ingredients.

The present invention is also concerned with the use of a acannabinoid-1 receptor antagonist/inverse agonist of Formula I and an anti Alzheimer's Disease agent for the manufacture of a medicament for the treatment of Alzheimer's Disease which comprises an effective amount of the cannabinoid-1 receptor antagonist/inverse agonist of Formula I and an effective amount of the anti Alzheimer's Disease agent, together or separately.

The present invention is also concerned with a product containing a cannabinoid-1 receptor antagonist/inverse agonist of Formula I and an anti Alzheimer's Disease agent as a combined preparation for simultaneous, separate or sequential use in treating Alzheimer's Disease.

The present invention also relates to the treatment of Alzheimer's Disease with a combination of a cannabinoid-1 receptor antagonist/inverse agonist and an anti Alzheimer's Disease agent which may be administered separately, the invention also relates to combining separate pharmaceutical combinations into a kit form. The kit, according to this invention, comprises two separate pharmaceutical compositions: a first unit dosage form comprising a prophylactically or therapeutically effective amount of a cannabinoid-1 receptor antagonist/inverse agonist of Formula I, or a pharmaceutically acceptable salt thereof, and a pharmaceutically acceptable carrier or diluent in a first unit dosage form, and a second unit dosage form comprising a prophylactically or therapeutically effective amount of an anti Alzheimer's Disease agent, or a pharmaceutically acceptable salt thereof, and a pharmaceutically acceptable carrier or diluent in a second unit dosage form and a container.

### DETAILED DESCRIPTION OF THE INVENTION

The present invention relates to methods of treating or preventing Alzheimer's Disease and Alzheimer's Disease related disorders comprising administering a therapeutically or prophylactically effective amount of a compound of Formula I

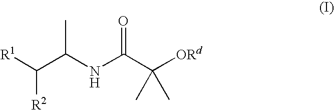

or a pharmaceutically acceptable salt thereof, wherein;
R¹ is selected from:
(1) cycloheteroalkyl,
(2) aryl,
(3) heteroaryl, and
(4) —NR^{a}R^{c},
   wherein aryl and heteroaryl are optionally substituted with one to three substituents independently selected from R^{b};
   R² is selected from:
(1) C₁₋₁₀ alkyl,
(2) C₃₋₁₀cycloalkyl-C₁₋₄alkyl,
(3) aryl-C₁₋₄alkyl, and
(4) heteroaryl-C₁₋₄alkyl,
   wherein each cycloalkyl, aryl and heteroaryl is optionally substituted with one to three substituents independently selected from R^{b};
   each R^{a} is independently selected from:
(1) hydrogen,
(2) methyl, and
(3) —CF₃;
   each R^{b} is independently selected from:
(1) halogen,
(2) cyano,
(3) trifluoromethyl,
(4) trifluoromethoxy,
(5) C₁₋₃alkyloxy, and
(6) C₁₋₃alkyl;
   R^{c} is independently selected from:
(1) hydrogen,
(2) C₁₋₆alkyl,
(3) aryl,
(4) heteroaryl,
(5) aryl-methyl, and
(6) heteroaryl-methyl,
   each R^{c} may be unsubstituted or substituted with one to three substituents selected from R^{h};
   R^{d} is independently selected from:
(1) cycloalkyl,
(2) aryl, and
(3) heteroaryl,
   each R^{d} may be unsubstituted or substituted with one to three substituents selected from R^{h};
   each R^{h} is independently selected from:
(1) halogen,
(2) C₁₋₃alkyl,
(3) —CN, and
(4) —CF₃,
   wherein when pyridyl groups are unsubstituted on the nitrogen, they are optionally present as the N-oxide.

In one embodiment of the present invention, R¹ is selected from:
(1) phenyl,
(2) pyridyl,
(3) indolyl,
(4) 7-aza-indolyl,
(5) thiophenyl, and
(6)

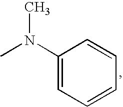

wherein each aryl and heteroaryl is optionally substituted with one or two substitutents independently selected from R^{b}, and each pyridyl is optionally present as the N-oxide.

In one class of this embodiment of the present invention, R¹ is selected from:
(1) phenyl,
(2) 3-cyanophenyl,
(3) 3-methylphenyl,
(4) 3,5-difluorophenyl,
(5) 3-pyridyl,
(6) 5-chloro-3-pyridyl,
(7) 5-methyl-3-pyridyl,
(8) 5-cyano-3-pyridyl,
(9) 1-oxido-5-cyano-3-pyridyl,
(10) 1-indolyl,
(11) 7-aza-indol-N-yl,
(12) 2-thiophenyl, and
(13)

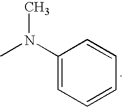

In a subclass of this class of the present invention, R¹ is 5-cyano-3-pyridyl.

In another embodiment of the present invention, R² is selected from:
(1) C₁₋₆alkyl,
(2) C₃₋₆cycloalkylmethyl,
(3) phenylmethyl, and
(4) heteroarylmethyl,
   wherein each cycloalkyl, phenyl and heteroaryl is optionally substituted with one to three substituents independently selected from R^{b}.

In one class of this embodiment of the present invention, R² is selected from:
(1) C₁₋₆alkyl,
(2) C₄₋₆cycloalkylmethyl,
(3) phenylmethyl, and
(4) pyridyl,
   wherein each cycloalkyl, phenyl and heteroaryl is optionally substituted with one or two substituents independently selected from R^{b}.

In a subclass of this class of the present invention, R² is selected from:
(1) 2-methylpropyl,
(2) n-pentyl,
(3) cyclobutylmethyl,
(4) cyclopentylmethyl,
(5) cyclohexylmethyl,
(6) benzyl,
(7) 4-chlorobenzyl,
(8) 4-methylbenzyl,
(9) 4-fluorobenzyl,
(10) 4-methoxybenzyl, and
(11) (5-chloro-2-pyridyl)methyl.

In one embodiment of the present invention, each R^{a} is independently selected from:
(1) hydrogen,
(2) methyl, and
(3) —CF₃.

In one class of this embodiment of the present invention, each R^{a} is independently selected from:
(1) hydrogen, and
(2) methyl.

In one embodiment of the present invention, each R^{b} is independently selected from:
(1) halogen,
(2) cyano,
(3) C₁₋₃ alkyloxy, and
(4) C₁₋₃ alkyl.

In one class of this embodiment of the present invention, each R^{b} is independently selected from:
(1) fluoro,
(2) chloro,
(3) bromo,
(4) iodo,
(5) cyano,
(6) methoxy, and
(7) methyl.

In one subclass of this class, each R^{b} is independently selected from:
(1) fluoro,
(2) chloro,
(3) cyano,
(4) methoxy, and
(5) methyl.

In one embodiment of the present invention, each R^{c} is independently selected from:
(1) hydrogen,
(2) C₁₋₆alkyl,
(3) phenyl,
(4) pyridyl,
(5) benzyl, and
(6) pyridyl-methyl;
   each R^{c} may be unsubstituted or substituted with a substituent selected from R^{h}.

In one class, R^{c} is phenyl.

In one embodiment of the present invention, R^{d} is selected from:
(1) C₄₋₆cycloalkyl,
(2) aryl, and
(3) heteroaryl,
   wherein R^{d} may be unsubstituted or substituted with one or two substituents selected from R^{h}.

In one class of the present invention, R^{d} is selected from:
(1) phenyl,
(2) pyridyl, and
(3) pyrimidinyl,
   wherein R^{d} may be unsubstituted or substituted with one or two substituents selected from R^{h}.

In one subclass of the present invention, R^{d} is selected from:
(1) phenyl,
(2) 4-chlorophenyl,
(3) 3-chlorophenyl,
(4) 3,5-difluorophenyl,
(5) 3,5-dichlorophenyl,
(6) 2-pyridyl,
(7) 5-chloro-2-pyridyl,
(8) 6-methyl-2-pyridyl,
(9) 5-trifluoromethyl-2-pyridyl,
(10) 4-trifluoromethyl-2-pyridyl,
(11) 4-trifluoromethyl-2-pyrimidyl, and
(12) 6-trifluoromethyl-4-pyrimidyl.
   In another subclass of the present invention, R^{d} is 5-trifluoromethyl-2-pyridyl.

In one embodiment of the present invention, each R^{h} is independently selected from:
(1) halogen,
(2) C₁₋₃alkyl,
(3) —CN, and
(4) —CF₃.

In one class of this embodiment, each R^{h} is independently selected from:
(1) fluoro,
(2) chloro,
(3) methyl,
(4) —CN, and
(5) —CF₃.

Particular novel compounds which may be employed in the methods, uses and compositions of the present invention, include:
(1) N-[3-(4-chlorophenyl)-1-methyl-2-phenylpropyl]-2-(4-chlorophenyloxy)-2-methylpropanamide;
(2) N-[3-(4-chlorophenyl)-1-methyl-2-phenylpropyl]-2-(2-pyridyloxy)-2-methylpropanamide;
(3) N-[3-(4-chlorophenyl)-1-methyl-2-(3-pyridyl)propyl]-2-(4-chlorophenyloxy)-2-methylpropanamide;
(4) N-[3-(4-chlorophenyl)-1-methyl-2-phenylpropyl]-2-(3,5-difluorophenyloxy)-2-methylpropanamide;
(5) N-[3-(4-chlorophenyl)-2(S)-phenyl-1(S)-methylpropyl]-2-(3,5-dichlorophenyloxy)-2-methylpropanamide;
(6) N-[3-(4-chlorophenyl)-1-methyl-2-phenylpropyl]-2-(3-chlorophenyloxy)-2-methylpropanamide;
(7) N-[3-(4-chlorophenyl)-2-(3,5-difluorophenyl)-1-methylpropyl]-2-(2-pyridyloxy)-2-methylpropanamide;
(8) N-[(2(S),3(S))-3-(4-chlorophenyl)-1-methyl-2-phenyl-propyl]-2-(5-chloro-2-pyridyloxy)-2-methylpropanamide;
(9) N-[3-(4-chlorophenyl)-1-methyl-2-phenylpropyl]-2-(6-methyl-pyridyloxy)-2-methylpropanamide;
(10) N-[3-(4-chlorophenyl)-1-methyl-2-phenylpropyl]-2-(phenyloxy)-2-methylpropanamide;
(11) N-[(3-(4-chlorophenyl)-1-methyl-2-phenylpropyl]-2-(5-trifluoromethylpyridyloxy)-2-methylpropanamide;
(12) N-[3-(4-chlorophenyl)-2-(3-pyridyl)-1-methylpropyl]-2-(5-trifluoromethyl-2-pyridyloxy)-2-methylpropanamide;
(13) N-[3-(4-chlorophenyl)-2-(3-cyanophenyl)-1-methylpropyl]-2-(5-trifluoromethyl-2-pyridyloxy)-2-methylpropanamide;
(14) N-[3-(4-chlorophenyl)-2-(5-chloro-3-pyridyl)-1-methylpropyl]-2-(5-trifluoromethyl-2-pyridyloxy)-2-methylpropanamide;
(15) N-[3-(4-chlorophenyl)-2-(5-methyl-3-pyridyl)-1-methylpropyl]-2-(5-trifluoromethyl-2-pyridyloxy)-2-methylpropanamide;
(16) N-[3-(4-chlorophenyl)-2-(5-cyano-3-pyridyl)-1-methylpropyl]-2-(5-trifluoromethyl-2-pyridyloxy)-2-methylpropanamide;
(17) N-[3-(4-chlorophenyl)-2-(3-methylphenyl)-1-methylpropyl]-2-(5-trifluoromethyl-2-pyridyloxy)-2-methylpropanamide;
(18) N-[3-(4-chlorophenyl)-2-phenyl-1-methylpropyl]-2-(4-trifluoromethyl-2-pyridyloxy)-2-methylpropanamide;
(19) N-[3-(4-chlorophenyl)-2-phenyl-1-methylpropyl]-2-(4-trifluoromethyl-2-pyrimidyloxy)-2-methylpropanamide;
(20) N-[3-(4-chlorophenyl)-1-methyl-2-(thiophen-3-yl)propyl]-2-(5-chloro-2-pyridyloxy)-2-methylpropanamide;
(21) N-[3-(5-chloro-2-pyridyl)-2-phenyl-1-methylpropyl]-2-(5-trifluoromethyl-2-pyridyloxy)-2-methylpropanamide;
(22) N-[3-(4-methyl-phenyl)-1-methyl-2-phenylpropyl]-2-(5-trifluoromethyl-phenyloxy)-2-methylpropanamide;
(23) N-[3-(4-fluoro-phenyl)-2-(3-cyano-phenyl)-1-methylpropyl]-2-(5-trifluoromethyl-2-pyridyloxy)-2-methylpropanamide;
(24) N-[3-(4-chlorophenyl)-2-(1-indolyl)-1-methyl)propyl]-2-(5-trifluoromethyl-2-oxypyridine-2-yl)-2-methylpropanamide;
(25) N-[3-(4-chlorophenyl)-2-(7-azaindol-N-yl)-1-methyl)propyl]-2-(5-trifluoromethyl-2-pyridyloxy)-2-methylpropanamide;
(26) N-[3-(4-chloro-phenyl)-2-(1-indolinyl)-1-methylpropyl]-2-(5-trifluoromethyl-2-pyridyloxy)-2-methylpropanamide;
(27) N-[3-(4-chloro-phenyl)-2-(N-methyl-anilino)-1-methylpropyl]-2-(5-trifluoromethyl-2-pyridyloxy)-2-methylpropanamide;
(28) N-[3-(4-methoxy-phenyl)-2-(3-cyano-phenyl)-1-methylpropyl]-2-(5-trifluoromethyl-2-pyridyloxy)-2-methylpropanamide;
(29) N-[3-(4-chlorophenyl)-2-(3-cyanophenyl)-1-methylpropyl]-2-(6-trifluoromethyl-4-pyrimidyloxy)-2-methylpropanamide;
(30) N-[2-(3-cyanophenyl)-1,4-dimethylpentyl]-2-(5-trifluoromethyl-2-pyridyloxy)-2-methylpropanamide;
(31) N-[3-(4-chlorophenyl)-2-(1-oxido-5-cyano-3-pyridyl]-1-methylpropyl]-2-(5-trifluoromethyl-2-pyridyloxy)-2-methylpropanamide;
(32) N-[2-(3-cyanophenyl)-3-cyclobutyl-1-methylpropyl]-2-(5-trifluoromethyl-2-pyridyloxy)-2-methylpropanamide;
(33) N-[2-(3-cyanophenyl)-1-methyl-heptyl]-2-(5-trifluoromethyl-2-pyridyloxy)-2-methylpropanamide;
(34) N-[2-(3-cyanophenyl)-3-cyclopentyl-1-methylpropyl]-2-(5-trifluoromethyl-2-pyridyloxy)-2-methylpropanamide;
(35) N-[2-(3-cyanophenyl)-3-cyclohexyl-1-methylpropyl]-2-(5-trifluoromethyl-2-pyridyloxy)-2-methylpropanamide;
   and pharmaceutically acceptable salts thereof.

In another embodiment of the present invention, novel compounds which may be employed in the methods, uses and compositions of the present invention, include:
(1) N-[(3-(4-chlorophenyl)-1-methyl-2-phenylpropyl]-2-(5-trifluoromethylpyridyloxy)-2-methylpropanamide;
(2) N-[3-(4-chlorophenyl)-2-(3-pyridyl)-1-methylpropyl]-2-(5-trifluoromethyl-2-pyridyloxy)-2-methylpropanamide;
(3) N-[3-(4-chlorophenyl)-2-(3-cyanophenyl)-1-methylpropyl]-2-(5-trifluoromethyl-2-pyridyloxy)-2-methylpropanamide;
(4) N-[3-(4-chlorophenyl)-2-(5-chloro-3-pyridyl)-1-methylpropyl]-2-(5-trifluoromethyl-2-pyridyloxy)-2-methylpropanamide;
(5) N-[3-(4-chlorophenyl)-2-(5-methyl-3-pyridyl)-1-methylpropyl]-2-(5-trifluoromethyl-2-pyridyloxy)-2-methylpropanamide;
(6) N-[3-(4-chlorophenyl)-2-(5-cyano-3-pyridyl)-1-methylpropyl]-2-(5-trifluoromethyl-2-pyridyloxy)-2-methylpropanamide;
(7) N-[3-(4-chlorophenyl)-2-(3-methylphenyl)-1-methylpropyl]-2-(5-trifluoromethyl-2-pyridyloxy)-2-methylpropanamide;
(8) N-[3-(5-chloro-2-pyridyl)-2-phenyl-1-methylpropyl]-2-(5-trifluoromethyl-2-pyridyloxy)-2-methylpropanamide;
(9) N-[3-(4-fluoro-phenyl)-2-(3-cyano-phenyl)-1-methylpropyl]-2-(5-trifluoromethyl-2-pyridyloxy)-2-methylpropanamide;
(10) N-[3-(4-chlorophenyl)-2-(1-indolyl)-1-methyl)propyl]-2-(5-trifluoromethyl-2-oxypyridine-2-yl)-2-methylpropanamide;
(11) N-[3-(4-chlorophenyl)-2-(7-azaindol-N-yl)-1-methyl)propyl]-2-(5-trifluoromethyl-2-pyridyloxy)-2-methylpropanamide;
(12) N-[3-(4-chloro-phenyl)-2-(1-indolinyl)-1-methylpropyl]-2-(5-trifluoromethyl-2-pyridyloxy)-2-methylpropanamide;
(13) N-[3-(4-chloro-phenyl)-2-(N-methyl-anilino)-1-methylpropyl]-2-(5-trifluoromethyl-2-pyridyloxy)-2-methylpropanamide;
(14) N-[3-(4-methoxy-phenyl)-2-(3-cyano-phenyl)-1-methylpropyl]-2-(5-trifluoromethyl-2-pyridyloxy)-2-methylpropanamide;
(15) N-[2-(3-cyanophenyl)-1,4-dimethylpentyl]-2-(5-trifluoromethyl-2-pyridyloxy)-2-methylpropanamide;
(16) N-[3-(4-chlorophenyl)-2-(1-oxido-5-cyano-3-pyridyl]-1-methylpropyl]-2-(5-trifluoromethyl-2-pyridyloxy)-2-methylpropanamide;
(17) N-[2-(3-cyanophenyl)-3-cyclobutyl-1-methylpropyl]-2-(5-trifluoromethyl-2-pyridyloxy)-2-methylpropanamide;
(18) N-[2-(3-cyanophenyl)-1-methyl-heptyl]-2-(5-trifluoromethyl-2-pyridyloxy)-2-methylpropanamide;
(19) N-[2-(3-cyanophenyl)-3-cyclopentyl-1-methylpropyl]-2-(5-trifluoromethyl-2-pyridyloxy)-2-methylpropanamide; and
(20) N-[2-(3-cyanophenyl)-3-cyclohexyl-1-methylpropyl]-2-(5-trifluoromethyl-2-pyridyloxy)-2-methylpropanamide;
   and pharmaceutically acceptable salts thereof.

In another embodiment of the present invention, novel compounds which may be employed in the methods, uses and compositions of the present invention, include:
(1) N-[3-(4-chlorophenyl)-1-methyl-2-phenylpropyl]-2-(4-chlorophenyloxy)-2-methylpropanamide;
(2) N-[3-(4-chlorophenyl)-1-methyl-2-phenylpropyl]-2-(2-pyridyloxy)-2-methylpropanamide;
(3) N-[3-(4-chlorophenyl)-1-methyl-2-(3-pyridyl)propyl]-2-(4-chlorophenyloxy)-2-methylpropanamide;
(4) N-[3-(4-chlorophenyl)-1-methyl-2-phenylpropyl]-2-(3,5-difluorophenyloxy)-2-methylpropanamide;
(5) N-[3-(4-chlorophenyl)-1-methyl-2-phenylpropyl]-2-(3-chlorophenyloxy)-2-methylpropanamide;
(6) N-[(2(S),3(S))-3-(4-chlorophenyl)-1-methyl-2-phenyl-propyl]-2-(5-chloro-2-pyridyloxy)-2-methylpropanamide;
(7) N-[3-(4-chlorophenyl)-2-(3-pyridyl)-1-methylpropyl]-2-(5-trifluoromethyl-2-pyridyloxy)-2-methylpropanamide;
(8) N-[3-(4-chlorophenyl)-2-(3-cyanophenyl)-1-methylpropyl]-2-(5-trifluoromethyl-2-pyridyloxy)-2-methylpropanamide;
(9) N-[3-(4-chlorophenyl)-2-(5-chloro-3-pyridyl)-1-methylpropyl]-2-(5-trifluoromethyl-2-pyridyloxy)-2-methylpropanamide;
(10) N-[3-(4-chlorophenyl)-2-(5-methyl-3-pyridyl)-1-methylpropyl]-2-(5-trifluoromethyl-2-pyridyloxy)-2-methylpropanamide;
(11) N-[3-(4-chlorophenyl)-2-(5-cyano-3-pyridyl)-1-methylpropyl]-2-(5-trifluoromethyl-2-pyridyloxy)-2-methylpropanamide;
(12) N-[3-(4-chlorophenyl)-2-(3-methylphenyl)-1-methylpropyl]-2-(5-trifluoromethyl-2-pyridyloxy)-2-methylpropanamide;
   and pharmaceutically acceptable salts thereof.

In another embodiment of the present invention, novel compounds which may be employed in the methods, uses and compositions of the present invention, include:
(1) N-[3-(4-chlorophenyl)-2(S)-phenyl-1(S)-methylpropyl]-2-(3,5-dichlorophenyloxy)-2-methylpropanamide;
(2) N-[3-(4-chlorophenyl)-2-(3,5-difluorophenyl)-1-methylpropyl]-2-(2-pyridyloxy)-2-methylpropanamide;
(3) N-[3-(4-chlorophenyl)-1-methyl-2-phenylpropyl]-2-(6-methyl-pyridyloxy)-2-methylpropanamide;
(4) N-[3-(4-chlorophenyl)-1-methyl-2-phenylpropyl]-2-(phenyloxy)-2-methylpropanamide;
(5) N-[(3-(4-chlorophenyl)-1-methyl-2-phenylpropyl]-2-(5-trifluoromethylpyridyloxy)-2-methylpropanamide;
(6) N-[3-(4-chlorophenyl)-2-phenyl-1-methylpropyl]-2-(4-trifluoromethyl-2-pyridyloxy)-2-methylpropanamide;
(7) N-[3-(4-chlorophenyl)-2-phenyl-1-methylpropyl]-2-(4-trifluoromethyl-2-pyrimidyloxy)-2-methylpropanamide;
(8) N-[3-(4-chlorophenyl)-1-methyl-2-(thiophen-3-yl)propyl]-2-(5-chloro-2-pyridyloxy)-2-methylpropanamide;
(9) N-[3-(5-chloro-2-pyridyl)-2-phenyl-1-methylpropyl]-2-(5-trifluoromethyl-2-pyridyloxy)-2-methylpropanamide;
(10) N-[3-(4-methyl-phenyl)-1-methyl-2-phenylpropyl]-2-(5-trifluoromethyl-phenyloxy)-2-methylpropanamide;
(11) N-[3-(4-fluoro-phenyl)-2-(3-cyano-phenyl)-1-methylpropyl]-2-(5-trifluoromethyl-2-pyridyloxy)-2-methylpropanamide;
(12) N-[3-(4-chlorophenyl)-2-(1-indolyl)-1-methyl)propyl]-2-(5-trifluoromethyl-2-oxypyridine-2-yl)-2-methylpropanamide;
(13) N-[3-(4-chlorophenyl)-2-(7-azaindol-N-yl)-1-methyl)propyl]-2-(5-trifluoromethyl-2-pyridyloxy)-2-methylpropanamide;
(14) N-[3-(4-chloro-phenyl)-2-(1-indolinyl)-1-methylpropyl]-2-(5-trifluoromethyl-2-pyridyloxy)-2-methylpropanamide;
(15) N-[3-(4-chloro-phenyl)-2-(N-methyl-anilino)-1-methylpropyl]-2-(5-trifluoromethyl-2-pyridyloxy)-2-methylpropanamide;
(16) N-[3-(4-methoxy-phenyl)-2-(3-cyano-phenyl)-1-methylpropyl]-2-(5-trifluoromethyl-2-pyridyloxy)-2-methylpropanamide;
(17) N-[3-(4-chlorophenyl)-2-(3-cyanophenyl)-1-methylpropyl]-2-(6-trifluoromethyl-4-pyrimidyloxy)-2-methylpropanamide;
(18) N-[2-(3-cyanophenyl)-1,4-dimethylpentyl]-2-(5-trifluoromethyl-2-pyridyloxy)-2-methylpropanamide;
(19) N-[3-(4-chlorophenyl)-2-(1-oxido-5-cyano-3-pyridyl]-1-methylpropyl]-2-(5-trifluoromethyl-2-pyridyloxy)-2-methylpropanamide;
(20) N-[2-(3-cyanophenyl)-3-cyclobutyl-1-methylpropyl]-2-(5-trifluoromethyl-2-pyridyloxy)-2-methylpropanamide;
(21) N-[2-(3-cyanophenyl)-1-methyl-heptyl]-2-(5-trifluoromethyl-2-pyridyloxy)-2-methylpropanamide;
(22) N-[2-(3-cyanophenyl)-3-cyclopentyl-1-methylpropyl]-2-(5-trifluoromethyl-2-pyridyloxy)-2-methylpropanamide; and
(23) N-[2-(3-cyanophenyl)-3-cyclohexyl-1-methylpropyl]-2-(5-trifluoromethyl-2-pyridyloxy)-2-methylpropanamide;
   and pharmaceutically acceptable salts thereof.

In another embodiment of the present invention, novel compounds which may be employed in the methods, uses and compositions of the present invention, include:
(1) N-[3-(4-chlorophenyl)-2-(3-pyridyl)-1-methylpropyl]-2-(5-trifluoromethyl-2-pyridyloxy)-2-methylpropanamide;
(2) N-[3-(4-chlorophenyl)-2-(5-chloro-3-pyridyl)-1-methylpropyl]-2-(5-trifluoromethyl-2-pyridyloxy)-2-methylpropanamide;
(3) N-[3-(4-chlorophenyl)-2-(5-methyl-3-pyridyl)-1-methylpropyl]-2-(5-trifluoromethyl-2-pyridyloxy)-2-methylpropanamide;
(4) N-[3-(4-chlorophenyl)-2-(5-cyano-3-pyridyl)-1-methylpropyl]-2-(5-trifluoromethyl-2-pyridyloxy)-2-methylpropanamide;
(5) N-[3-(4-chlorophenyl)-2-(3-methylphenyl)-1-methylpropyl]-2-(5-trifluoromethyl-2-pyridyloxy)-2-methylpropanamide;
   and pharmaceutically acceptable salts thereof.

In yet another embodiment of the present invention, novel compounds which may be employed in the methods, uses and compositions of the present invention, include:
(1) N-[(3-(4-chlorophenyl)-1-methyl-2-phenylpropyl]-2-(5-trifluoromethylpyridyloxy)-2-methylpropanamide;
(2) N-[3-(4-chlorophenyl)-2-(3-cyanophenyl)-1-methylpropyl]-2-(5-trifluoromethyl-2-pyridyloxy)-2-methylpropanamide;
(3) N-[3-(5-chloro-2-pyridyl)-2-phenyl-1-methylpropyl]-2-(5-trifluoromethyl-2-pyridyloxy)-2-methylpropanamide;
(4) N-[3-(4-fluoro-phenyl)-2-(3-cyano-phenyl)-1-methylpropyl]-2-(5-trifluoromethyl-2-pyridyloxy)-2-methylpropanamide;
(5) N-[3-(4-chlorophenyl)-2-(1-indolyl)-1-methyl)propyl]-2-(5-trifluoromethyl-2-oxypyridine-2-yl)-2-methylpropanamide;
(6) N-[3-(4-chlorophenyl)-2-(7-azaindol-N-yl)-1-methyl)propyl]-2-(5-trifluoromethyl-2-pyridyloxy)-2-methylpropanamide;
(7) N-[3-(4-chloro-phenyl)-2-(1-indolinyl)-1-methylpropyl]-2-(5-trifluoromethyl-2-pyridyloxy)-2-methylpropanamide;
(8) N-[3-(4-chloro-phenyl)-2-(N-methyl-anilino)-1-methylpropyl]-2-(5-trifluoromethyl-2-pyridyloxy)-2-methylpropanamide;
(9) N-[3-(4-methoxy-phenyl)-2-(3-cyano-phenyl)-1-methylpropyl]-2-(5-trifluoromethyl-2-pyridyloxy)-2-methylpropanamide;
(10) N-[2-(3-cyanophenyl)-1,4-dimethylpentyl]-2-(5-trifluoromethyl-2-pyridyloxy)-2-methylpropanamide;
(11) N-[3-(4-chlorophenyl)-2-(1-oxido-5-cyano-3-pyridyl]-1-methylpropyl]-2-(5-trifluoromethyl-2-pyridyloxy)-2-methylpropanamide;
(12) N-[2-(3-cyanophenyl)-3-cyclobutyl-1-methylpropyl]-2-(5-trifluoromethyl-2-pyridyloxy)-2-methylpropanamide;
(13) N-[2-(3-cyanophenyl)-1-methyl-heptyl]-2-(5-trifluoromethyl-2-pyridyloxy)-2-methylpropanamide;
(14) N-[2-(3-cyanophenyl)-3-cyclopentyl-1-methylpropyl]-2-(5-trifluoromethyl-2-pyridyloxy)-2-methylpropanamide; and
(15) N-[2-(3-cyanophenyl)-3-cyclohexyl-1-methylpropyl]-2-(5-trifluoromethyl-2-pyridyloxy)-2-methylpropanamide;
   and pharmaceutically acceptable salts thereof.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like.

“Cycloalkyl” means mono- or bicyclic or bridged saturated carbocyclic rings, each of which having from 3 to 10 carbon atoms. Examples of cycloalkyl include cyclopropyl, cyclobutyl, cyclopentyl, cyclohexyl, cycloheptyl, and the like.

“Aryl” means mono- or bicyclic aromatic rings containing only carbon atoms. Examples of aryl include phenyl, naphthyl, and the like.

“Heteroaryl” means a mono- or bicyclic aromatic ring containing at least one heteroatom selected from N, O and S, with each ring containing 5 to 6 atoms. Examples of heteroaryl include pyrrolyl, isoxazolyl, isothiazolyl, pyrazolyl, pyridyl, 7-aza-indolyl, thiophenyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, imidazothiazolyl, and the like. In particular, “heteroaryl” includes pyridyl, pyrimidyl, and thiophenyl. The heteroaryl ring may be substituted on one or more carbon or nitrogen atoms

“Cycloheteroalkyl” means mono- or bicyclic or bridged saturated rings containing at least one heteroatom selected from N, S and O, each of said ring having from 3 to 10 atoms in which the point of attachment may be carbon or nitrogen. The term also includes monocyclic heterocycle fused to an aryl or heteroaryl group in which the point of attachment is on the non-aromatic portion. Examples of “cycloheteroalkyl” include indolyl, azaindolyl and the like. The cycloheteroalkyl ring may be substituted on the ring carbons and/or the ring nitrogens.

“Halogen” includes fluorine, chlorine, bromine and iodine.

When any variable (e.g., R¹, R^{d}, etc.) occurs more than one time in any constituent or in formula I, its definition on each occurrence is independent of its definition at every other occurrence. Also, combinations of substituents and/or variables are permissible only if such combinations result in stable compounds.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to

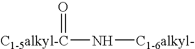

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

### Optical Isomers—Diastereomers—Geometric Isomers—Tautomers

Compounds of Formula I, I-35 and II may contain one or more asymmetric centers and can thus occur as racemates and racemic mixtures, single enantiomers, diastereomeric mixtures and individual diastereomers. The present invention is meant to comprehend all such isomeric forms of the compounds of Formula I, I-35 and II.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I, I-35 and II.

Compounds of the Formula I, I-35 and II may be separated into diastereoisomeric pairs of enantiomers by, for example, fractional crystallization from a suitable solvent, for example MeOH or EtOAc or a mixture thereof. The pair of enantiomers thus obtained may be separated into individual stereoisomers by conventional means, for example by the use of an optically active amine as a resolving agent or on a chiral HPLC column.

Alternatively, any enantiomer of a compound of the general Formula I and Formulas I-35 and II may be obtained by stereospecific synthesis using optically pure starting materials or reagents of known configuration.

It is generally preferable to administer compounds of the present invention as enantiomerically pure formulations. Racemic mixtures can be separated into their individual enantiomers by any of a number of conventional methods. These include chiral chromatography, derivatization with a chiral auxillary followed by separation by chromatography or crystallization, and fractional crystallization of diastereomeric salts.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to the compounds of Formula I, I-35 and II are meant to also include the pharmaceutically acceptable salts.

### Utilities

Compounds of the present invention are modulators of the CB1 receptor. In particular, the compounds of structural formula I, I-35 and II are antagonists or inverse agonists of the CB1 receptor.

An “agonist” is a compound (hormone, neurotransmitter or synthetic compound) which binds to a receptor, inducing a conformational change in the receptor which, in turn, produces a response such as contraction, relaxation, secretion, change in enzyme activity, etc. similar to that elicited by the physiologically relevant agonist ligand(s) for that receptor. An “antagonist” is a compound which attenuates the effect of an agonist. An “inverse agonist” is a compound which acts on a receptor but produces the opposite effect produced by the agonist of the particular receptor.

The compounds of this invention are modulators of the CB1 receptor and as such are useful as centrally acting drugs in treating, ameliorating, controlling Alzheimer's Disease or reducing the risk of Alzheimer's Disease.

The compounds of this invention may also be useful for the treating, preventing, controlling, ameliorating or reducing the risk of an Alzheimer's Disease related disorders.

The compounds of this invention are also useful as centrally acting drugs in the treatment of psychosis, dementia, age related cognitive decline, mild cognitive impairment, memory impairment, aphasia, apraxia, agnosia and disturbances in executing functioning, cerebral amyloid angiopathy, multi-infarct dementia, dementia publistica, Down syndrome, memory deficits, memory impairment, and cognitive disorders, memory deficits, memory impairment, cognitive disorders, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, schizophrenia, non alcoholic fatty liver, non alcoholic steatohepatitis, Huntington's Disease, and addictive disorders. For example, the compounds may be useful for the treatment and prevention of dementia of the Alzheimer's type, multi-infarct dementia, dementia publistica, as well as for the treatment and prevention of early stage, intermediate stage or late stage dementia of the Alzheimer's type.

The method of treatment of this invention comprises a method of treating Alzheimer's Disease, or Alzheimer's Disease related disorders, by administering to a patient in need of such treatment or at risk of developing Alzheimer's disease a therapeutically effective or prophylactically effective amount of a compound of formula I, I-35 or II; or a pharmaceutically acceptable salt thereof.

Another aspect of the present invention is a method of treating Alzheimer's disease or an Alzheimer's Disease related disorder comprising administration to a patient in need of such treatment a therapeutically effective amount of the compound of formula I, or a pharmaceutically acceptable salt thereof.

Another aspect of the present invention is a method of treating Alzheimer's disease or an Alzheimer's Disease related disorder comprising administration to a patient in need of such treatment a therapeutically effective amount of the compound of formula I-35

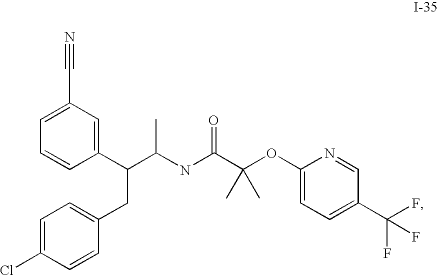

or a pharmaceutically acceptable salt thereof.

Another aspect of the present invention provides a method of treating Alzheimer's disease or an Alzheimer's Disease related disorder comprising administration to a patient in need of such treatment a therapeutically effective amount of the compound of formula II

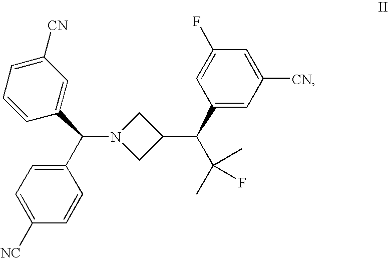

or a pharmaceutically acceptable salt thereof.

Another aspect of the present invention provides the use of a compound of formula I-35

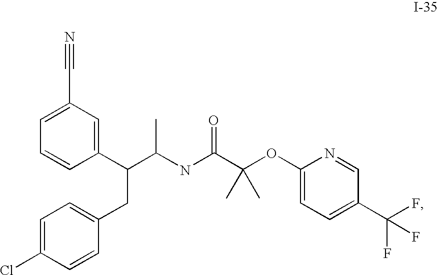

or a pharmaceutically acceptable salt thereof, for the manufacture of a medicament useful for the treatment of an Alzheimer's disease related disorder in a human patient in need of such treatment.

Another aspect of the present invention provides the use of a compound of formula II

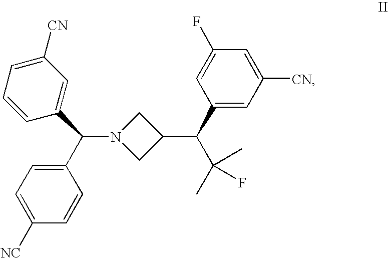

or a pharmaceutically acceptable salt thereof, for the manufacture of a medicament useful for the treatment of an Alzheimer's disease related disorder in a human patient in need of such treatment.

Another aspect of the present invention provides for the manufacture of a medicament for the treatment of age-related cognitive decline or mild cognitive impairment. A favored outcome of the treatment is prevention or delay of the onset of Alzheimer's disease. Such prevention or delay may be evidenced by halting or slowing of the patient's cognitive decline, or halting or slowing of the progress of cognitive impairment.

Another aspect of the present invention provides the use of a compound of formula I, or a pharmaceutically acceptable salt thereof, for the manufacture of a medicament for preventing or delaying the onset of dementia associated with Alzheimer's disease in a patient with age related cognitive decline or in a patient with mild cognitive impairment. Another aspect of the present invention provides the use of a compound of formula I-35, or a pharmaceutically acceptable salt thereof, for the manufacture of a medicament for preventing or delaying the onset of dementia associated with Alzheimer's disease in a patient with age related cognitive decline or in a patient with mild cognitive impairment. Another aspect of the present invention provides the use of a compound of formula II, or a pharmaceutically acceptable salt thereof, for the manufacture of a medicament for preventing or delaying the onset of dementia associated with Alzheimer's disease in a patient with age related cognitive decline or in a patient with mild cognitive impairment.

Another aspect of the present invention provides a method of treating age-related cognitive decline or mild cognitive impairment comprising administering to a patient in need thereof a therapeutically-effective amount of a compound of formula I, or a pharmaceutically acceptable salt thereof. Another aspect of the present invention provides a method of treating age-related cognitive decline or mild cognitive impairment comprising administering to a patient in need thereof a therapeutically-effective amount of a compound of formula I-35, or a pharmaceutically acceptable salt thereof. Another aspect of the present invention provides a method of treating age-related cognitive decline or mild cognitive impairment comprising administering to a patient in need thereof a therapeutically-effective amount of a compound of formula II, or a pharmaceutically acceptable salt thereof.

Another aspect of the present invention provides a method of preventing, retarding or arresting any further age-related cognitive decline or progression of mild cognitive impairment comprising administering to a patient in need thereof a therapeutically-effective amount of a compound of formula I, or a pharmaceutically acceptable salt thereof. Another aspect of the present invention provides a method of preventing, retarding or arresting any further age-related cognitive decline or progression of mild cognitive impairment comprising administering to a patient in need thereof a therapeutically-effective amount of a compound of formula I-35, or a pharmaceutically acceptable salt thereof. Another aspect of the present invention provides a method of preventing, retarding or arresting any further age-related cognitive decline or progression of mild cognitive impairment comprising administering to a patient in need thereof a therapeutically-effective amount of a compound of formula II, or a pharmaceutically acceptable salt thereof.

Another aspect of the present invention provides a method of preventing or delaying the onset of Alzheimer's disease comprising administering to a patient suffering from age related cognitive decline or mild cognitive impairment a therapeutically-effective amount of a compound of formula I, or a pharmaceutically acceptable salt thereof. Another aspect of the present invention provides a method of preventing or delaying the onset of Alzheimer's disease comprising administering to a patient suffering from age related cognitive decline or mild cognitive impairment a therapeutically-effective amount of a compound of formula I-35, or a pharmaceutically acceptable salt thereof. Another aspect of the present invention provides a method of preventing or delaying the onset of Alzheimer's disease comprising administering to a patient suffering from age related cognitive decline or mild cognitive impairment a therapeutically-effective amount of a compound of formula II, or a pharmaceutically acceptable salt thereof.

Another aspect of the present invention provides a method of preventing or delaying the onset of dementia associated with Alzheimer's disease comprising administering to a patient suffering from age-related cognitive decline or mild cognitive impairment a therapeutically-effective amount of a compound of formula I, or a pharmaceutically acceptable salt thereof. Another aspect of the present invention provides a method of preventing or delaying the onset of dementia associated with Alzheimer's disease comprising administering to a patient suffering from age-related cognitive decline or mild cognitive impairment a therapeutically-effective amount of a compound of formula I-35, or a pharmaceutically acceptable salt thereof. Another aspect of the present invention provides a method of preventing or delaying the onset of dementia associated with Alzheimer's disease comprising administering to a patient suffering from age-related cognitive decline or mild cognitive impairment a therapeutically-effective amount of a compound of formula II, or a pharmaceutically acceptable salt thereof.

Another aspect of the present invention provides a method of administering a compound of formula I, or a pharmaceutically acceptable salt thereof, to a person having mild cognitive impairment. Another aspect of the present invention provides a method of administering a compound of formula I-35, or a pharmaceutically acceptable salt thereof, to a person having mild cognitive impairment. Another aspect of the present invention provides a method of administering a compound of formula II, or a pharmaceutically acceptable salt thereof, to a person having mild cognitive impairment.

Another aspect of the present invention provides a method of administering a compound of formula I, or a pharmaceutically acceptable salt thereof, to a person exhibiting age-related cognitive decline. Another aspect of the present invention provides a method of administering a compound of formula I-35, or a pharmaceutically acceptable salt thereof, to a person exhibiting age-related cognitive decline. Another aspect of the present invention provides a method of administering a compound of formula II, or a pharmaceutically acceptable salt thereof, to a person exhibiting age-related cognitive decline.

Another aspect of the present invention provides methods for preventing, retarding or arresting the accumulation of insoluble Aβ in the brain of a patient suffering from age-related cognitive decline or mild cognitive impairment.

The medicaments useful in the invention are particularly suitable for administration to patients who suffer impaired memory function but do not exhibit other symptoms that would constitute dementia, such as aphasia, apraxia, agnosia or disturbance in executive functioning. Such impairment of memory function typically is not attributable to systemic or cerebral disease, such as stroke or metabolic disorders caused by pituitary dysfunction. Such patients may be in particular people aged 55 or over, especially people aged 60 or over, and preferably people aged 65 or over. Such patients may have normal patterns and levels of growth hormone secretion for their age. However, such patients may possess one or more additional risk factors for developing Alzheimer's disease. Such factors include a family history of the disease; a genetic predisposition to the disease; elevated serum cholesterol; adult-onset diabetes mellitus; raised CSF levels of total tau; raised CSF levels of phospho-tau; and lowered CSF levels of Aβ42.

In another aspect of the present invention, provides a method of administering a compound of formula I, I-35 or II, or a pharmaceutically acceptable salt thereof, is administered to a patient suffering from age-related cognitive decline or mild cognitive impairment who additionally possesses one or more risk factors for developing Alzheimer's Disease selected from: a family history of the disease; a genetic predisposition to the disease; elevated serum cholesterol; adult-onset diabetes mellitus; raised CSF levels of total tau; raised CSF levels of phospho-tau; and lowered CSF levels of Aβ42.

A genetic predisposition (especially towards early onset Alzheimer's disease) can arise from point mutations in one or more of the APP, presenilin-1 and presenilin-2 genes. Also, subjects who are homozygous for the ε4 isoform of the apolipoprotein E gene are at greater risk of developing AD.

The patient's degree of cognitive decline or impairment is advantageously assessed at regular intervals before, during and/or after a course of treatment with the compound of formula I, I-35 or II, or a pharmaceutically acceptable salt thereof, so that changes therein may be detected, e.g. the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med., 12 (1982), 397-408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment.

According to another aspect of the present invention there is provided the use of a compound of formula I, I-35 or II, or a pharmaceutically acceptable salt thereof, for the manufacture of a medicament for the treatment or prevention of Alzheimer's Disease or an Alzheimer's Disease related disorder. In another or alternative aspect of the present invention, there is therefore provided a product comprising a compound of formula I, I-35 or II, or a pharmaceutically acceptable salt thereof, for use in the treatment or prevention of Alzheimer's Disease or an Alzheimer's Disease related disorder.

Another aspect of the present invention provides a composition comprising a compound of formula I, I-35 or II, or a pharmaceutically acceptable salt thereof, and an anti-Alzheimer's disease agent, or a pharmaceutically acceptable salts thereof.

Another aspect of the present invention provides a method of treating Alzheimer's Disease, or Alzheimer's Disease related disorders, by administering to a patient in need of such treatment or at risk of developing Alzheimer's disease a therapeutically effective or prophylactically effective amount of a composition comprising a compound of formula I, I-35 or II, or a pharmaceutically acceptable salt thereof, and an anti-Alzheimer's disease agent, or a pharmaceutically acceptable salt thereof.

According to a further aspect of the present invention there is provided the use of a composition or combination of a compound of formula I, I-35 or II and an anti Alzheimer's disease agent for the manufacture of a medicament for the treatment or prevention of a Alzheimer's Disease or an Alzheimer's Disease related disorder. In a further or alternative aspect of the present invention, there is therefore provided a product comprising a combination of a compound of formula I, I-35 or II and an anti-Alzheimer's disease agent for use in the treatment or prevention of Alzheimer's Disease or an Alzheimer's Disease related disorder.

The term “Alzheimer's Disease” is described in the Diagnostic and Statistical Manual of Mental Disorders, 4^{th} ed., published by the American Psychiatric Association (DSM-IV) (e.g. pages 139-143). Diagnostic criteria for Alzheimer's disease includes the development of multiple cognitive deficits in a patient manifested by both (1) memory impairment (impaired ability to learn new information or to recall previously learned information), and (2) one (or more) of the following cognitive disturbances (a) aphasia (language disturbance), (b) apraxia (impaired ability to carry out motor activities despite intact motor function), (c) agnosia (failure to recognize or identify objects despite intact sensory function) and (d) disturbances in executing functioning (i.e. planning, organizing, sequencing, abstracting). Such cognitive deficits are not characterized as being due to any of the following: (1) other central nervous system conditions that cause progressive deficits in memory and cognition (e.g. cerebrovascular disease, Parkinson's disease, Huntington's disease, subdural hematoma, normal-pressure hydrocephalus, brain tumor), (2) systemic conditions that are known to cause dementia (e.g. hypothyroidism, vitamin B12 or folic acid deficiency, niacin deficiency, hypercalcemia, neurosyphilis, HIV infection), (3) substance-induced conditions.

As used herein, the term “Alzheimer's Disease” is meant to encompass all of the above definitions of Alzheimer's Disease.

Alzheimer's Disease-related disorders are associated with, caused by, or result from Alzheimer's Disease or preceed Alzheimer's Disease. Examples of Alzheimer's Disease-related disorders include, but are not limited to, dementia, age related cognitive decline, mild cognitive impairment, memory impairment, aphasia, apraxia, agnosia and disturbances in executing functioning, memory deficits, memory impairment, and cognitive disorders. In one embodiment of the present invention, Alzheimer's Disease related disorders are selected from: dementia, age related cognitive decline, mild cognitive impairment, memory impairment, aphasia, apraxia, agnosia and disturbances in executing functioning. In another embodiment of the present invention, Alzheimer's Disease related disorders are selected from: dementia, age related cognitive decline, and mild cognitive impairment. In another embodiment of the present invention, the Alzheimer's Disease related disorder is dementia.

Age-related cognitive decline and mild cognitive impairment (MCI) are conditions in which a memory deficit is present, but other diagnostic criteria for dementia are absent (Santacruz and Swagerty, American Family Physician, 63 (2001), 703-13). (See also “The ICD-10 Classification of Mental and Behavioural Disorders”, Geneva: World Health Organisation, 1992, 64-5). When used herein, age-related cognitive decline is characterized by a decline of at least six months' duration in at least one of: memory and learning; attention and concentration; thinking; language; and visuospatial functioning and a score of more than one standard deviation below the norm on standardized neuropsychologic testing such as the MMSE. In particular, there may be a progressive decline in memory. In the more severe condition mild cognitive impairment (MCI), the degree of memory impairment is outside the range considered normal for the age of the patient but Alzheimer's disease (AD) is not present. The differential diagnosis of MCI and mild AD is described by Petersen et al., Arch. Neurol., 56 (1999), 303-8. In the same article, Petersen et al. disclose that the patients suffering from MCI typically experience a progressive increase in cognitive impairment and in many cases develop AD. Further information on the differential diagnosis of MCI is provided by Knopman et al, Mayo Clinic Proceedings, 78 (2003), 1290-1308. In a study of elderly subjects, Tuokko et al (Arch, Neurol., 60 (2003) 577-82) found that those exhibiting MCI at the outset had a three-fold increased risk of developing dementia within 5 years.

Grundman et al (J. Mol. Neurosci., 19 (2002), 23-28) report that lower baseline hippocampal volume in MCI patients is a prognostic indicator for subsequent AD. Similarly, Andreasen et al (Acta Neurol. Scand, 107 (2003) 47-51) report that high CSF levels of total tau, high CSF levels of phospho-tau and lowered CSF levels of Aβ42 are all associated with increased risk of progression from MCI to AD.

Age-related cognitive decline and mild cognitive impairment are distinct from the significant cognitive deficit that sometimes results from cerebral or systemic diseases and traumas, such as stroke, concussion, or major disfunction of the pituitary

“Treatment” (of Alzheimer's Disease and Alzheimer's Disease-related disorders) refers to the administration of the compounds or compositions of the present invention to treat Alzheimer's Disease and to treat early, intermediate and late stage dementia of Alzheimer's type. One outcome of treatment may be treating Alzheimer's Disease. Another outcome of treatment may be controlling Alzheimer's Disease. Another outcome of treatment may be ameliorating Alzheimer's disease. Another outcome of treatment may be reducing the risk of Alzheimer's Disease. Another outcome of treatment may be the prevention of the dementia of Alzheimer's type. Another outcome of treatment may be the prevention of the early stage dementia of Alzheimer's type. Another outcome of treatment may be the prevention of intermediate stage dementia of Alzheimer's type. Another outcome of treatment may be the prevention of late stage dementia of Alzheimer's type. Another outcome of treatment may be treating an Alzheimer's Disease related disorder. Another outcome of treatment may be prevention or delay of the onset of Alzheimer's disease. Another outcome of treatment may be halting or slowing of the patient's cognitive decline. Another outcome of treatment may be halting or slowing of the progress of cognitive impairment. Another outcome of treatment may be enhanced clearance of Aβ from the brain. Another outcome of treatment may be preventing or delaying the onset of dementia associated with Alzheimer's disease. Another outcome of treatment may be treating age-related cognitive decline or mild cognitive impairment. Another outcome of treatment may be preventing, retarding or arresting any further age-related cognitive decline or progression of mild cognitive impairment. Another outcome of treatment may be preventing or delaying the onset of Alzheimer's disease. Another outcome of treatment may be preventing or delaying the onset of dementia associated with Alzheimer's disease. Another outcome of treatment may be preventing, retarding or arresting the accumulation of insoluble Aβ in the brain. Moreover, if treatment is commenced in early stage dementia of Alzheimer's type in Alzheimer's disease subjects, such treatment may prevent the occurrence, progression or severity of Alzheimer's disease and/or Alzheimer's disease related disorders. Treatment includes prevention of Alzheimer's disease and Alzheimer's disease related disorders.

“Prevention” (of Alzheimer's Disease and Alzheimer's Disease-related disorders) refers to the administration of the compounds or compositions of the present invention to prevent Alzheimer's Disease and to prevent early, intermediate and late stage dementia of Alzheimer's type. One outcome of prevention may be reducing the risk of Alzheimer's Disease. Another outcome of prevention may be the prevention of the dementia of Alzheimer's type. Another outcome of prevention may be the prevention of the early stage dementia of Alzheimer's type. Another outcome of prevention may be the prevention of intermediate stage dementia of Alzheimer's type. Another outcome of prevention may be the prevention of late stage dementia of Alzheimer's type. Another outcome of prevention may be prevention of Alzheimer's Disease related disorders. Another outcome of prevention may be prevention or delay of the onset of Alzheimer's disease. Another outcome of prevention may be halting or slowing of the patient's cognitive decline. Another outcome of prevention may be halting or slowing of the progress of cognitive impairment. Another outcome of prevention may be preventing or delaying the onset of dementia associated with Alzheimer's disease. Another outcome of prevention may be preventing, retarding or arresting any further age-related cognitive decline or progression of mild cognitive impairment. Another outcome of prevention may be preventing or delaying the onset of Alzheimer's disease. Another outcome of prevention may be preventing the onset of dementia associated with Alzheimer's disease. Another outcome of prevention may be preventing or retarding the accumulation of insoluble Aβ in the brain.

The terms “administration of” and or “administering a” compound should be understood to mean providing a compound of the invention or a prodrug of a compound of the invention to the individual in need of treatment.

The administration of the compound of structural formula I, I-35 or II in order to practice the present methods of therapy is carried out by administering an effective amount of the compound of structural formula I, I-35 or II to the patient in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The utilities of the present compounds in these diseases or disorders may be demonstrated in animal disease models that have been reported in the literature. The following are examples of such animal disease models: a) tone-shock association shows slowing of extinction of associative memory (Marsicano, Nature, vol. 418, 1 Aug. 2002); b) 8-arm radial maze error rate reduction and spatial memory enhancement (Lichtman, European Journal of Pharmacology 404 (2000) 175-179; Wolff, European Journal of Pharmacology 477 (2003) 213-217); c) olfactory memory of social recognition and improvement of short term memory (Terranova, Psychopharmacology (1996) 126: 165-172; d) β-amyloid amnesia step-through passive avoidance (Mazzola, European Journal of Pharmacology, 477 (2003) 219-225); e) KO increases LTP (Bohme et al., Neuroscience Vol. 95, No. 1, 5-7 (2000)); and f) Elevated T-maze use in reversal of scopolamine induced deficit (Takahashi, Neuroscience Letters 380 (2005) 270-275).

### Dose Ranges

The magnitude of prophylactic or therapeutic dose of a compound of Formula I, I-35 or II will, of course, vary with the nature of the severity of the condition to be treated and with the particular compound of Formula I, I-35 or II and its route of administration. It will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.001 mg to about 25 mg (preferably from 0.01 mg to about 1 mg) of a compound of Formula I, I-35 or II per kg of body weight per day and for preventive use from about 0.1 mg to about 100 mg (preferably from about 1 mg to about 100 mg and more preferably from about 1 mg to about 10 mg) of a compound of Formula I, I-35 or II per kg of body weight per day.

In the case where an oral composition is employed, a suitable dosage range is, e.g. from about 0.01 mg to about 1000 mg of a compound of Formula I, I-35 or II per day, preferably from about 0.1 mg to about 10 mg per day. For oral administration, the compositions are preferably provided in the form of tablets containing from 0.01 to 1,000 mg, preferably 0.01, 0.05, 0.1, 0.5, 1, 2.5, 5, 10, 15, 20, 25, 30, 40, 50, 100, 250, 500, 750 or 1000 milligrams of the active ingredient for the symptomatic adjustment of the dosage to the patient to be treated.

### Pharmaceutical Compositions

Another aspect of the present invention provides pharmaceutical compositions which comprises a compound of Formula I, I-35 or II and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, I-35 or II, additional active ingredient(s), and pharmaceutically acceptable excipients.

Any suitable route of administration may be employed for providing a mammal, especially a human, with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I, I-35 or II as an active ingredient or a pharmaceutically acceptable salt thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (aerosol inhalation), sublingual, or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I, I-35 or II in suitable propellants, such as fluorocarbons or hydrocarbons and dry powder inhalation (DPI) aerosol, which may be formulated as a dry powder of a compound of Formula I, I-35 or II with or without additional excipients.

Suitable topical formulations of a compound of formula I, I-35 or II include transdermal devices, aerosols, creams, solutions, ointments, gels, lotions, dusting powders, and the like. The topical pharmaceutical compositions containing the compounds of the present invention ordinarily include about 0.005% to 5% by weight of the active compound in admixture with a pharmaceutically acceptable vehicle. Transdermal skin patches useful for administering the compounds of the present invention include those well known to those of ordinary skill in that art. To be administered in the form of a transdermal delivery system, the dosage administration will, of course, be continuous rather than intermittent throughout the dosage regimen.

In practical use, the compounds of Formula I, I-35 or II can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, liquid filled capsules, and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

Pharmaceutical compositions of the present invention suitable for oral administration may be presented as discrete units such as capsules (including timed release and sustained release formulations), pills, cachets, powders, granules or tablets each containing a predetermined amount of the active ingredient, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from 0.01 to 1,000 mg, particularly 0.01, 0.05, 0.1, 0.5, 1, 2.5, 3, 5, 6, 10, 15, 25, 50, 75, 100, 125, 150, 175, 180, 200, 225, 500, 750 and 1,000 milligrams of the active ingredient for the symptomatic adjustment of the dosage to the patient to be treated, and each cachet or capsule contains from about 0.01 to 1,000 mg, particularly 0.01, 0.05, 0.1, 0.5, 1.0, 2.5, 3, 5, 6, 10, 15, 25, 50, 75, 100, 125, 150, 175, 180, 200, 225, 500, 750 and 1,000 milligrams of the active ingredient for the symptomatic adjustment of the dosage to the patient to be treated.

Additional suitable means of administration of the compounds of the present invention include injection, intravenous bolus or infusion, intraperitoneal, subcutaneous, intramuscular and topical, with or without occlusion.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compounds of Formula I, I-35 or II:

| Injectable Suspension (I.M.) | mg/mL |
|---|---|
| Compound of Formula I, I-35 or II | 10.0 |
| Methylcellulose | 5.0 |
| Tween 80 | 0.5 |
| Benzyl alcohol | 9.0 |
| Benzalkonium chloride | 1.0 |

Water for injection to a total volume of 1 mL

| Tablet | mg/tablet |
|---|---|
| Compound of Formula I, I-35 or II | 25 |
| Microcrystalline Cellulose | 415 |
| Povidone | 14.0 |
| Pregelatinized Starch | 43.5 |
| Magnesium Stearate | 2.5 |
| | 500 |

| Capsule | mg/capsule |
|---|---|
| Compound of Formula I, I-35 or II | 25 |
| Lactose Powder | 573.5 |
| Magnesium Stearate | 1.5 |
| | 600 |

| Aerosol | Per canister | |
|---|---|---|
| Compound of Formula I, I-35 or II | 24 | mg |
| Lecithin, NF Liq. Conc. | 1.2 | mg |
| Trichlorofluoromethane, NF | 4.025 | g |
| Dichlorodifluoromethane, NF | 12.15 | g |

### Combination Therapy

Compounds of Formula I, I-35 and II may be used in combination with other drugs that are used in the treatment/prevention/suppression or amelioration of the diseases or conditions for which compounds of Formula I, I-35 and II are useful. Such other drugs may be administered, by a route and in an amount commonly used therefor, contemporaneously or sequentially with a compound of Formula I, I-35 or II. When a compound of Formula I, I-35 or II is used contemporaneously with one or more other drugs, a pharmaceutical composition containing such other drugs in addition to the compound of Formula I, I-35 or II is preferred. Accordingly, the pharmaceutical compositions of the present invention include those that also contain one or more other active ingredients, in addition to a compound of Formula I, I-35 or II. Examples of other active ingredients that may be combined with a compound of Formula I, I-35 or II include, but are not limited to: anti-Alzheimer's Disease agents, antipsychotic agents, cognition enhancing agents, anti-migraine agents, anti-inflammatory agents, and anti-Parkinson's agents, which may be administered separately or in the same pharmaceutical compositions.

It will be appreciated that for the treatment or prevention of Alzheimer's disease, and Alzheimer's Disease related disorders, a compound of the present invention may be used in conjunction with other anti-Alzheimer's Disease agents.

The present invention also provides a method for the treatment or prevention of Alzheimer's Disease or an Alzheimer's Disease related disorder, which method comprises administration to a patient in need of such treatment or at risk of developing Alzheimer's Disease or an Alzheimer's Disease related disorder an amount of a compound of this invention and an amount of an anti-Alzheimer's Disease agent, such that together they give effective relief.

Another aspect of the present invention provides a combination of a compound of formula I, I-35 or II and at least one anti-Alzheimer's disease agent which modifies the production or processing of Aβ in the brain, said at least one agent being selected from:

(a) compounds which inhibit the secretion of Aβ;

(b) compounds which selectively inhibit the secretion of the 1-42 isoform of Aβ;

(c) compounds which inhibit the aggregation of Aβ; and

(d) antibodies which selectively bind to Aβ;

for use in treatment or prevention of a disease associated with deposition of Aβ in the brain, wherein the disease is selected from Alzheimer's Disease, cerebral amyloid angiopathy, multi-infarct dementia, dementia publistica and Down syndrome.

Compounds which inhibit the secretion of Aβ, include but are not limited to, inhibitors of gamma secretase, inhibitors of beta secretase, compounds that inhibit the formation or release of Aβ, including GSK-3 inhibitors and particularly GSK-3 alpha inhibitors, such as lithium. Compounds which selectively inhibit the secretion of the 1-42 isoform of Aβ, include but are not limited to, NSAIDs and their analogs. Compounds which inhibit the aggregation of Aβ; including but are not limited to, chelating agents such as clioquinol.

Suitable anti-Alzheimer's Disease agents of use in combination with a compound of the present invention include, but are not limited to, 1) acetyl-cholinesterase inhibitors such as galantamine, rivastigmine, donepezil, tacrine, phenserine, ladostigil and ABT-089; 2) beta-secretase inhibitors, such as CTS21166; 3) glycine transport inhibitors, including the compounds disclosed in WO 03/037325, WO 03/030886, WO 03/006013, WO 03/006021, WO 03/006423, WO 03/006453, WO 02/002122, WO 01/70672, WO 02/02505, WO 02/02506, WO 02/02512, WO 02/02520, WO 02/098849 and WO 02/100820; 4) alpha 7 nicotinic agonists, such as SSR 180711, MEM3454 and MEM63908; 5) gamma-secretase inhibitors, such as GSI953, LY450139, LY411575, TAK 070, Tarenflurbil, and the compounds disclosed in WO 01/53255, WO 01/66564, WO 01/70677, WO 01/90084, WO 01/77144, WO 02/30912, WO 02/36555, WO 02/081435, WO 02/081433, WO 03/018543, WO 03/013506, WO 03/013527, WO 03/014075, WO 03/093252, WO 2004/031137, WO 2004/031138, WO 2004/031139, WO 2004/039800 and WO 2004/039370; 6) gamma secretase modulators, such as E2012; 7) tau phosphorylation inhibitors; 8) blockers of Aβ oligomer formation and/or inhibitors of Aβ aggregation such as clioquinol, DP-109, Apan™ (Praecis), Alzhemed™ (Neurochem), PTI-777 and PTI-00703 (ProteoTech), and the compounds disclosed in WO 99/16741, WO 96/28471, WO 98/08868, WO 00/052048, WO 00/064420, WO 03/017994, WO 99/59571, WO 00/149281, WO 96/39834, WO 01/83425, WO 01/55093, WO 00/76988, WO 00/76987, WO 00/76969, WO 00/76489, WO 97/26919, WO 97/16194 and WO 97/16191, WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621; 9) 5-HT4 agonists, such as PRX 03140; 10) 5HT6 antagonists, such as GSK 742457, SGS-518, SAM315, E6795, SL-65.0155, SRA-333 and xaliproden; 11) p25/CDK5 inhibitors; 12) HMG-CoA reductase inhibitors; 13) NK1/NK3 receptor antagonists; 14) NSAID's and NSAID derivatives including ibuprofen, sulindac sulfide, flufenamic acid, flurbiprofen, fenoprofen, mefenamic acid, indomethacin, (R)-flurbiprofen, HCT-1026, NCX-2216, and compounds disclosed in WO 01/78721, US 2002/0128319, and WO 02/092072; 15) vitamin E; 16) anti-amyloid antibodies, (including anti-amyloid humanized monoclonal antibodies), such as bapineuzumab, AAB002, RN1219, ACC001, CAD106 and AZD3102, and the compounds disclosed in WO 99/60024 and WO 00/72880; 17) 5-HT1A antagonists, such as lecozotan; 18) COX-2 inhibitors; 19) anti-inflammatory agents, such as (R)-flurbiprofen, nitroflurbiprofen, rosiglitazone, ND-1251, VP-025, HT-0712, EHT-202, and HF0220; 20) CB-1 receptor antagonists or other CB-1 receptor inverse agonists, such as AVE 1625, and rimonabant, and those disclosed in WO 03/077847, WO 04/012671, WO 05/000809, US2004-0058820, US2006-0293299, and US2005-0272763; 21) antibiotics such as doxycycline and rifampin; 22) N-methyl-D-aspartate (NMDA) receptor antagonists, such as memantine, neramexane, dimebon, CX717, and EVT-101; 23) NR2B antagonists; 24) androgen receptor modulators; 25) mGluR5 modulators; 26) growth hormone and growth hormone secretagogues such as ibutamoren, ibutamoren mesylate, and capromorelin, and those disclosed in: U.S. Pat. No. 5,767,124, U.S. Pat. No. 5,536,716, WO 94/13696, EP 0615977B, U.S. Pat. No. 5,578,593; WO 01/04119, WO 98/25897, WO 98/10653, WO 97/36873, WO 97/34604, WO 97/15574, WO 97/11697, WO 96/32943, WO 96/13265, WO 96/02530, WO 95/34311, WO 95/14666, WO 95/13069, WO 94/19367, WO 94/05634 and WO 92/16524, EP 1002802A, EP 0995748A, WO 98/58948, WO 98/58947, WO 97/24369, WO 01/34593, WO 00/26252, WO 00/01726, WO 99/64456, WO 99/58501, WO 99/36431, WO 98/58950, WO 98/08492, WO 98/03473, WO 97/40071, WO 97/40023, WO 97/23508, WO 97/00894, WO 96/24587, WO 96/24580, WO 96/22997, WO 95/17423, WO 95/17422, WO 96/15148, WO 97/22620, WO 02/32888, WO 02/32878, WO 00/49037, WO 00/10565, WO 99/08699, WO 02/057241, WO 02/056873, WO 01/85695, WO 00/54729 and WO 00/24398; 27) histamine H₃ receptor antagonists, such as ABT-834, ABT-829, ABT-239, GSK 189254, GSK 239512, MK-0249, JNN17216498, BFs2649, and CEP16795; 28) AMPA agonists or AMPA modulators, such as CX717, LY404187 and S-18986; 29) PDE IV inhibitors, such as MEM141, HT0712, AVE8112 and the compounds disclosed in WO 03/108579, WO 02/060875, WO 02/074726, WO 02/098878, WO 01/46151, U.S. Pat. No. 5,449,686, U.S. Pat. No. 5,552,438, WO 98/45268 and WO 99/20625; 30) GABA_{A} inverse agonists; 31) GABA_{A} α5 receptor ligands; 32) GABA_{B} receptor ligands; 33) potassium channel blockers; 34) neuronal nicotinic agonists, such as ABT-089; 35) plasminogen activator inhibitors, such as PAZ417; 36) cathepsin B inhibitors; 37) GSK3β inhibitors, such as AZD1080, SAR502250 and CEP 16805; 38) selective M1 agonists; 39) neuronal nicotinic agonists; 40) microtubule affinity regulating kinase (MARK) ligands; 41) amyloid aggregation blockers and/or sequesters, such as ELND-005, AZD-103, TTP4000, PF449-4700, TTP-488, PBT-2, N251, and Alzhemed; 42) amyloid vaccine and monoclonal Ab, such as R1450, GSK933776A, MK-V950, AAB-001, M266/LY2062430 and PF4360365; 43) 5-HT modulators, such as PRX-07034/37, SYN-114, and SRA444; 44) AZD-1080, NGX-267, FGL-L, NP-12, T817, SP233, PF3084014, AL108, AC-1202, Paliproden/SR-57667, Leuprolide, GT-1061, SAM531, LX6171, AQW051, AVE8122, PYM-50028, ONO-2506, MEM-1003, SGS-742; 45) Aβ formation or release inhibitors, including those disclosed in WO 98/28268, WO 02/47671, WO 99/67221, WO 01/34639, WO 01/34571, WO 00/07995, WO 00/38618, WO 01/92235, WO 01/77086, WO 01/74784, WO 01/74796, WO 01/74783, WO 01/60826, WO 01/19797, WO 01/27108, WO 01/27091, WO 00/50391, WO 02/057252, US 2002/0025955 and US2002/0022621; 46) PPARα and/or PPARδ modulators, such as those disclosed in WO 02/100836; 47) antibodies which selectively bind to Aβ, including ADDLS (antibodies specific to Aβ-derived diffusable ligands) and the compounds disclosed in WO 03/016466, WO 03/016467, WO 03/015691, WO 01/62801, WO 2004/031400, WO 99/60024 and WO 00/72880; 48) the compounds disclosed in: U.S. Pat. No. 6,410,563, U.S. Pat. No. 6,743,802, U.S. Pat. No. 5,536,716, U.S. Pat. No. 6,984,669, U.S. Pat. No. 7,238,706, WO 03/066577, WO 04/048374, WO 03/018579, WO 05/087760, WO 03/093266, WO 06/096564, WO 05/070905, WO 04/020408, WO 05/077905, WO 05/013985, WO 05/047246, WO 04/108705, WO 02/068409, WO 07/011,833, WO 07/100,366, WO 03/018543, WO 03/093252, WO 06/055178, WO 06/0121656, and WO 07/007,890; 49) Tacrine (COGNEX®); 50) Rivastigmine (Novastis); 51) Galantamine (Shire) and 52) P-450 inhibitors, such as ritonavir; or other drugs that affect receptors or enzymes that either increase the efficacy, safety, convenience, or reduce unwanted side effects of the compounds of the present invention. The foregoing list of combinations is illustrative only and not intended to be limiting in any way.

The above compounds are only illustrative of the anti-Alzheimer's Disease agents that can be used in the compositions of the present invention. As this listing of compounds is not meant to be comprehensive, the methods of the present invention may employ any cannabinoid-1 receptor antagonist/inverse agonist of formula I, I-35 or II and any anti-Alzheimer's Disease agent, and are not limited to any particular structural class of compounds.

In a further aspect of the present invention, there is provided a pharmaceutical composition comprising a CB1 receptor modulator of this invention and one or more active ingredients, such as but not limited to an anti-Alzheimer's disease agent, together with at least one pharmaceutically acceptable carrier or excipient.

According to a further aspect of the present invention there is provided the use of a compound of formula I, I-35 or II and one or more active ingredients, such as but not limited to an anti-Alzheimer's disease agent, for the manufacture of a medicament for the treatment or prevention of a CB1 receptor modulator mediated disease, such as Alzheimer's Disease or an Alzheimer's disease related disorder. In a further or alternative aspect of the present invention, there is therefore provided a product comprising a CB1 receptor modulator and one or more active ingredients, such as but not limited to an anti-Alzheimer's disease agent, as a combined preparation for simultaneous, separate or sequential use in the treatment or prevention of CB1 receptor modulator mediated disease. Such a combined preparation may be, for example, in the form of a twin pack.

It will be appreciated that the CB1 receptor modulator and the anti-Alzheimer's Disease agent may be present as a combined preparation for simultaneous, separate or sequential use for the treatment or prevention of Alzheimer's Disease and/or an Alzheimer's Disease related disorder.

It will be appreciated that when using a combination of the present invention, the CB1 receptor modulator and the anti-Alzheimer's Disease agent may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, the anti Alzheimer's disease agent may be administered as a tablet and then, within a reasonable period of time, the CB1 receptor modulator may be administered either as an oral dosage form such as a tablet or a fast-dissolving oral dosage form. By a “fast-dissolving oral formulation” is meant, an oral delivery form which when placed on the tongue of a patient, dissolves within about 10 seconds.

It will be appreciated that a combination of a conventional anti-Alzheimer's Disease agent with a CB1 receptor modulator may provide an enhanced or synergistic effect in the treatment of Alzheimer's Disease and Alzheimer's Disease related disorders. Such a combination would be expected to provide for a rapid onset of action to treat Alzheimer's Disease symptoms thereby enabling prescription on an “as needed basis”. Furthermore, such a combination may enable a lower dose of the anti-Alzheimer's disease agent to be used without compromising the efficacy of the anti-Alzheimer's Disease agent, thereby minimizing the risk of adverse side-effects.

It will be understood that the scope of compositions of the compounds of this invention with other agents useful for treating or preventing Alzheimer's Disease and Alzheimer's Disease related disorders includes in principle any combination with any pharmaceutical composition useful for treating Alzheimer's Disease and Alzheimer's Disease-related disorders.

The term “therapeutically effective amount” means the amount the compound of structural formula I, I-35 or II, alone or in combination, that will elicit the biological or medical response of a tissue, system, animal or human that is being sought by the researcher, veterinarian, medical doctor or other clinician, which includes alleviation of the symptoms of the disorder being treated. The novel methods of treatment of this invention are for disorders known to those skilled in the art. The term “mammal” includes humans.

The weight ratio of the compound of the Formula I, I-35 or II to the second active ingredient may be varied and will depend upon the effective dose of each ingredient. Generally, an effective dose of each will be used. Thus, for example, when a compound of the Formula I, I-35 or II is combined with an anti-Alzheimer's Disease agent the weight ratio of the compound of the Formula I to the anti-Alzheimer's Disease agent will generally range from about 1000:1 to about 1:1000, preferably about 200:1 to about 1:200. Combinations of a compound of the Formula I, I-35 or II and other active ingredients will generally also be within the aforementioned range, but in each case, an effective dose of each active ingredient should be used.

The CB-1 antagonists/inverse agonists of formula I, including I-35, and their preparation are disclosed in U.S. Pat. No. 6,972,295, and U.S. patent application Ser. No. 11/629,005; and International Publication No. WO 2003/077847; WO 2004/048317; and WO 2006/017045. The CB-1 antagonist/inverse agonist of formula II, and its preparation is disclosed in U.S. Patent Application Publication No. US2006-0293299; and International Publication No. WO 2005/000809. Specific compounds of formula I and the compound of II useful in the present invention include:

| Example | | | |
|---|---|---|---|
| No. | Name | Structure | |
| I-2 | N-[2,3-Bis(4- Chlorophenyl)-1- methylpropyl]-2-(4- chlorophenyl-oxy)- 2-methyl- propanamide | 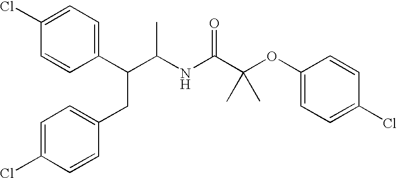 | |
| I-3 | N-[2,3-Bis(4- Chlorophenyl)-1- methylpropyl]-2-(4- chlorophenyl-oxy)- 2-methyl- propanamide | 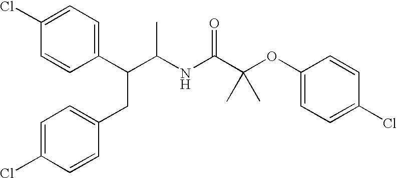 | |
| I-4 | N-[3-(4-Chloro- phenyl)-1-methyl-2- phenyl-propyl]-2- (3-chlorophenyl- oxy)-2-methyl- propanamide | 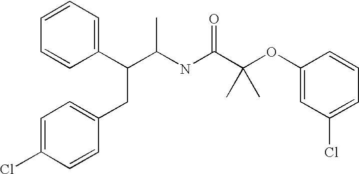 | |
| I-5 | N-[3-(4-Chloro- phenyl)-1-methyl-2- phenyl-propyl]-2- (3,5-difluoro- phenyloxy)-2- methylpropanamide | 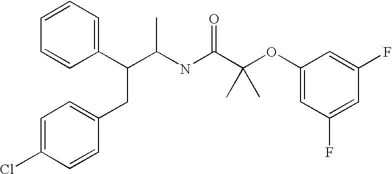 | |
| I-6 | N-[3-(4-Chloro- phenyl)-1-methyl-2- phenylpropyl]-2-(2- pyridyloxy)-2- methylpropanamide | 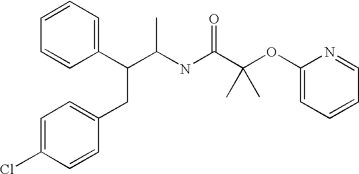 | |
| I-7 | N-[3-(4-Chloro- phenyl)-1-methyl-2- (3-pyridyl)propyl]- 2-(4-chlorophenyl- oxy)-2-methyl- propanamide | 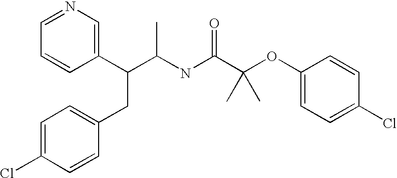 | |
| I-8 | N-[2,3-Bis(4- Chlorophenyl)-1- methylpropyl]-2-(4- chlorophenyloxy)- 2-methyl- propanamide | 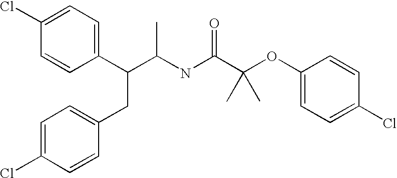 | |
| I-9 | N-[2,3-Bis(4- Chlorophenyl)-1- methylpropyl]-2-(4- chlorophenyloxy)- 2-methyl- propanamide | 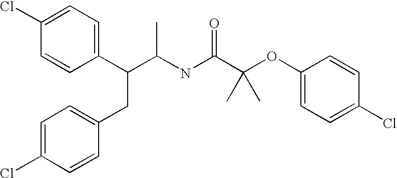 | |
| I-10 | N-[3-(4- Chlorophenyl)-1- methyl-2-phenyl- propyl]-2-(3- chlorophenyloxy)- 2-methyl- propanamide | 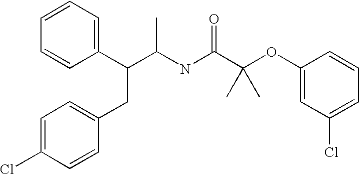 | |
| I-11 | N-[3-(4- Chlorophenyl)-1- methyl-2-phenyl- propyl]-2-(3- chlorophenyloxy)- 2-methyl- propanamide | 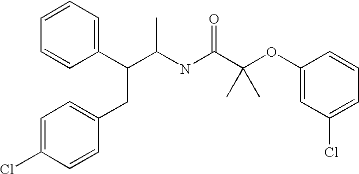 | |
| I-12 | N-[3-(4-Chloro- phenyl)-1-methyl-2- phenyl-propyl]-2- (3,5-difluorophenyl- oxy)-2-methyl- propanamide | 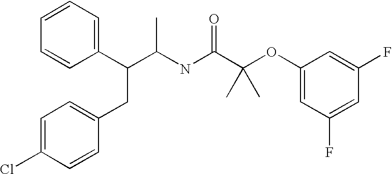 | |
| I-13 | N-[3-(4-Chloro- phenyl)-1-methyl-2- phenyl-propyl]-2- (3,5-difluorophenyl- oxy)-2-methyl- propanamide | 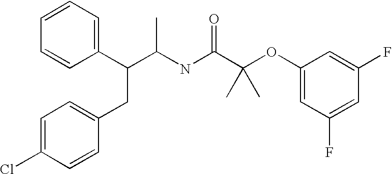 | |
| I-14 | N-[3-(4-Chloro- phenyl)-1-methyl-2- phenylpropyl]-2-(2- pyridyloxy)-2- methylpropanamide | 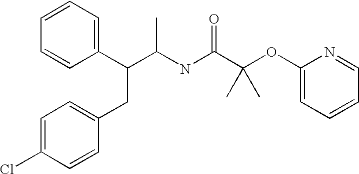 | |
| I-15 | N-[3-(4-Chloro- phenyl)-1-methyl-2- phenylpropyl]-2-(2- pyridyloxy)-2- methylpropanamide | 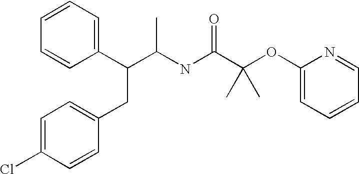 | |
| I-16 | N-[3-(4-Chloro- phenyl)-1-methyl-2- (3-pyridyl)propyl]- 2-(4-chlorophenyl- oxy)-2-methyl- propanamide | 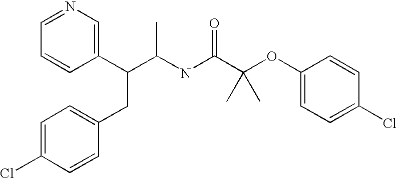 | |
| I-17 | N-[3-(4-Chloro- phenyl)-1-methyl-2- (3-pyridyl)propyl]- 2-(4-chlorophenyl- oxy)-2-methyl- propanamide | 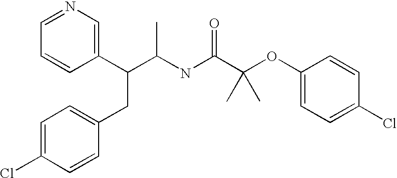 | Enan- tiomer B |
| I-18 | N-[3-(4-chloro- phenyl)-2(S)- phenyl-1(S)- methylpropyl]-2- (3,5-dichloro- phenyloxy)-2- methylpropanamide | 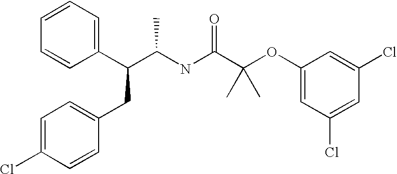 | |
| I-19 | N-[2,3-Bis(4- chlorophenyl)-1- methylpropyl]-2-(4- chlorophenylamino)- 2-methylpropan- amide | 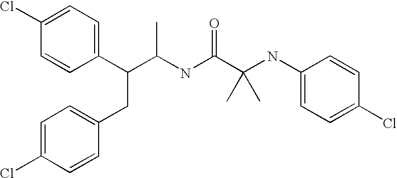 | |
| I-20 | N-(2,3-Diphenyl-1- methylpropyl)-2-(4- chlorophenoxy)-2- methylpropanamide (Diastereomer β) | 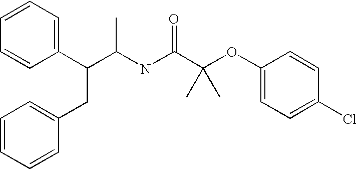 | |
| I-21 | N-[3-(4-chloro- phenyl)-1-methyl-2- phenyl-propyl]-2- (4-chlorophenyl- oxy)-2-methyl- propanamide | 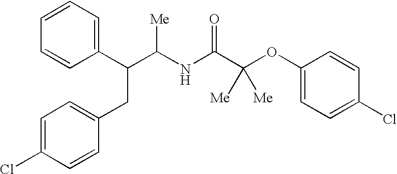 | |
| I-22 | N-(3-(4-chloro- phenyl)-2-phenyl- methylpropyl)-2- methyl-2-phenoxy- propanamide | 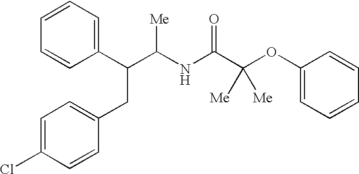 | |
| I-23 | N-[3-(4-Chloro- phenyl)-2-(3,5- difluorophenyl)-1- methylpropyl]-2- methyl-2-(2- pyridyloxy) propanamide | 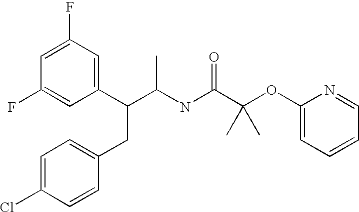 | Diaster- eomer α |
| I-24 | N-[3-(4-Chloro- phenyl)-2-(3,5- difluorophenyl)-1- methylpropyl]-2- methyl-2-(2- pyridyloxy)- propanamide | 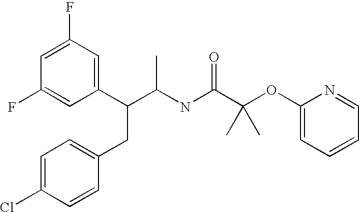 | Enan- tiomer A |
| I-25 | N-[3-(4-Chloro- phenyl)-2-(3,5- difluorophenyl)-1- methylpropyl]-2- methyl-2-(2- pyridyloxy)- propanamide | 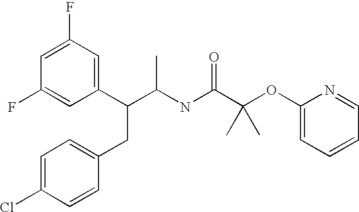 | Enan- tiomer B |
| I-26 | N-[(2S,3S)-3 -(4- Chlorophenyl)-1- methyl-2-phenyl- propyl]-2-(5- chloropyridyloxy)- 2-methyl- propanamide | 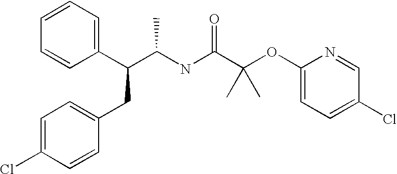 | |
| I-27 | N-[(2S,3S)-3-(4- Chlorophenyl)-1- methyl-2-phenyl- propyl]-2-(6- methylpyridyloxy)- 2-methyl- propanamide | 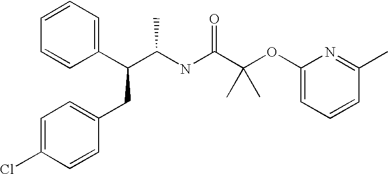 | |
| I-28 | N-[(2S,3S)-3-(4- Chlorophenyl)-1- methyl-2-phenyl- propyl]-2-(4- trifluoromethylphen yloxy)-2-methyl- propanamide | 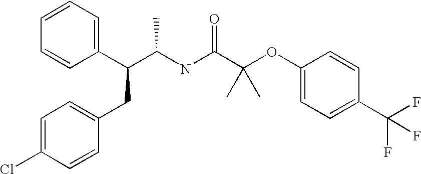 | |
| I-29 | N-[(2S,3S)-3-(4- Chlorophenyl)-1- methyl-2-phenyl- propyl]-2-(5- trifluoromethylpyrid yloxy)-2- methylpropanamide | 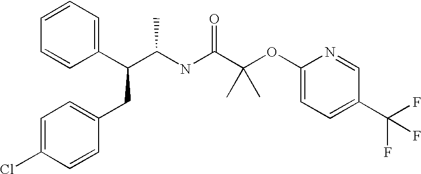 | |
| I-30 | N-[3-(5-chloro-2- pyridyl)-2(S)- phenyl-1(S)- methylpropyl]-2-(5- trifluoromethyl-2- pyridyloxy)-2- methylpropanamide | 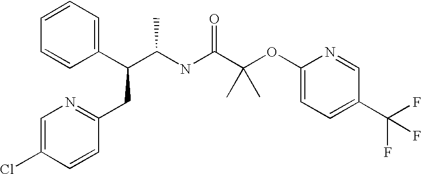 | |
| I-31 | N-[3-(4-chloro- phenyl)-2(S)- phenyl-1(S)- methylpropyl]-2-(4- trifluoromethyl-2- pyridyloxy)-2- methylpropanamide | 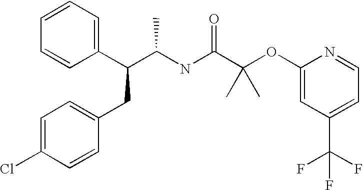 | |
| I-32 | N-[3-(4-chloro- phenyl)-2(S)- phenyl-1(S)- methylpropyl]-2-(4- trifluoromethyl-2- pyrimidyloxy)-2- methylpropanamide | 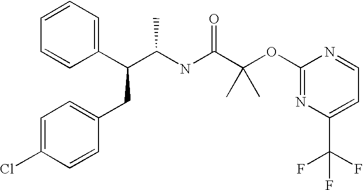 | |
| I-33 | N-[3-(4-chloro- phenyl)-2(S)- phenyl-1(S)- methylpropyl]-2-(4- trifluoromethyl-4- pyrimidyloxy)-2- methylpropanamide | 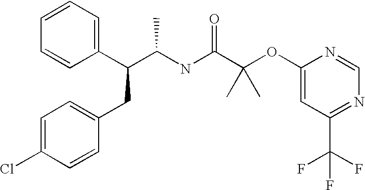 | |
| I-34 | N-[3-(4-Chloro- phenyl)-2-(3- methylphenyl)-1- methylpropyl]-2-(5- trifluoromethyl-2- pyridyloxy)-2- methylpropanamide | 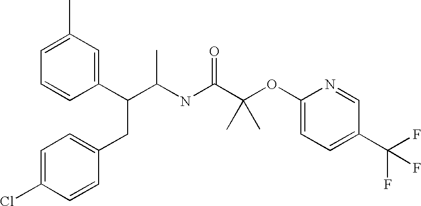 | Diaster- eomer α |
| I-35 | N-[3-(4-Chloro- phenyl)-2-(3- cyanophenyl)-1- methylpropyl]-2-(5- trifluoromethyl-2- pyridyloxy)-2- methylpropanamide | 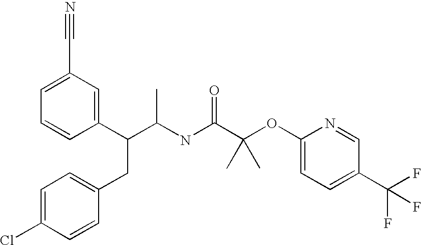 | Diaster- eomer α |
| I-36 | N-[3-(4-Chloro- phenyl)-2-(3- cyanophenyl)-1- methylpropyl]-2-(6- trifluoromethyl-4- pyrimidyloxy)-2- methylpropanamide | 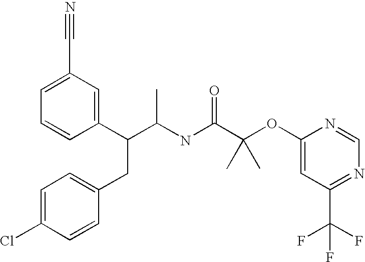 | Diaster- eomer α |
| I-37 | N-[3-(4-Chloro- phenyl)-2-(3- pyridyl)-1-methyl- propyl]-2-(5- trifluoromethyl-2- pyridyloxy)-2- methylpropanamide | 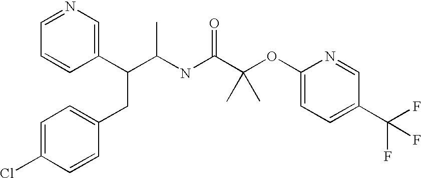 | Diaster- eomer α |
| I-38 | N-[3-(4-Chloro- phenyl)-2-(5- chloro-3-pyridyl)-1- methylpropyl]-2-(5- trifluoromethyl-2- pyridyloxy-2- methylpropanamide | 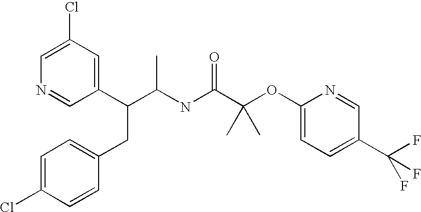 | Diaster- eomer α |
| I-39 | N-[3-(4-Chloro- phenyl)-2-(5-cyano- 3-pyridyl)-1- methylpropyl]-2-(5- trifluoromethyl-2- pyridyloxy)-2- methylpropanamide | 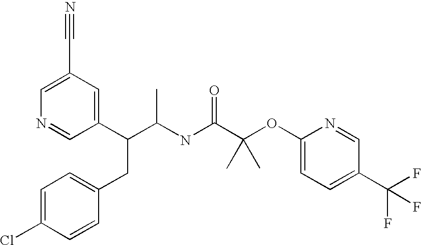 | Diaster- eomer α |
| I-40 | N-[3-(4-Chloro- phenyl)-2-(5- methyl-3-pyridyl)- 1-methylpropyl]-2- (5-trifluoromethyl- 2-pyridyloxy)-2- methylpropanamide | 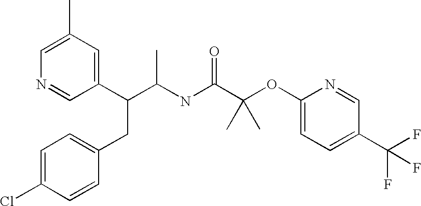 | Diaster- eomer α |
| I-41 | N-[3-(4-chloro- phenyl)-2-(3- methylphenyl)-1- methylpropyl]-2-(5- trifluoromethyl-2- pyridyloxy)-2- methylpropanamide | 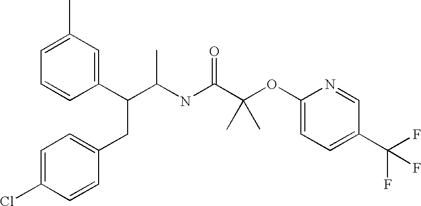 | Enan- tiomer A |
| I-42 | N-[3-(4-chloro- phenyl)-2-(3- methylphenyl)-1- methylpropyl]-2-(5- trifluoromethyl-2- pyridyloxy)-2- methylpropanamide | 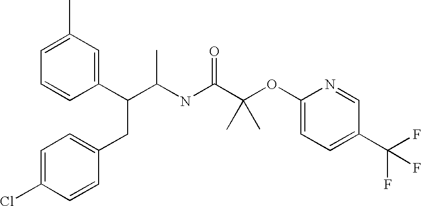 | Enan- tiomer B |
| I-43 | N-[3-(4-chloro- phenyl)-2-(3- cyanophenyl)-1- methylpropyl]-2-(5- trifluoromethyl-2- pyridyloxy)-2- methylpropanamide | 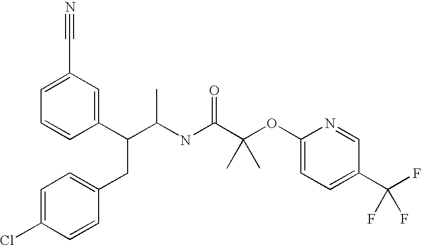 | Enan- tiomer A |
| I-44 | N-[3-(4-chloro- phenyl)-2-(3- cyanophenyl)-1- methylpropyl]-2-(5- trifluoromethyl-2- pyridyloxy)-2- methylpropanamide | 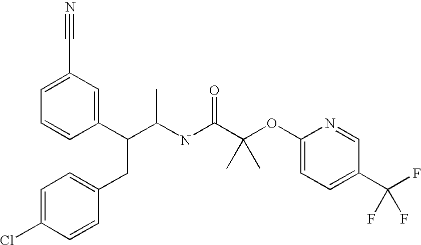 | Enan- tiomer B |
| I-45 | N-[3-(4-Chloro- phenyl)-2-(3- pyridyl)-1-methyl- propyl]-2-(5- trifluoromethyl-2- pyridyloxy)-2- methylpropanamide | 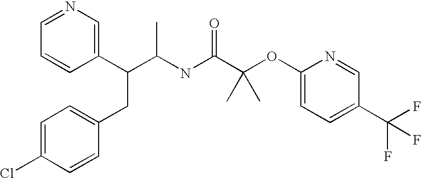 | Enan- tiomer A |
| I-46 | N-[3-(4-Chloro- phenyl)-2-(3- pyridyl)-1-methyl- propyl]-2-(5- trifluoromethyl-2- pyridyloxy)-2- methylpropanamide | 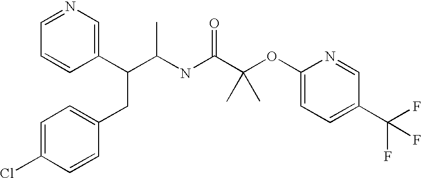 | Enan- tiomer B |
| I-47 | N-[3-(4-Chloro- phenyl)-2-(5- chloro-3-pyridyl)-1- methylpropyl]-2-(5- trifluoromethyl-2- pyridyloxy)-2- methylpropanamide | 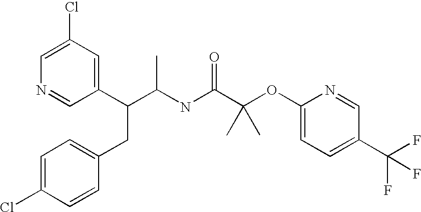 | Enan- tiomer A |
| I-48 | N-[3-(4-Chloro- phenyl)-2-(5- chloro-3-pyridyl)-1- methylpropyl]-2-(5- trifluoromethyl-2- pyridyloxy)-2- methylpropanamide | 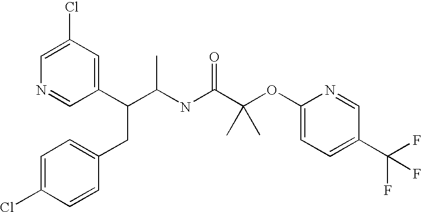 | Enan- tiomer B |
| I-49 | N-[3-(4-Chloro- phenyl)-2-(5-cyano- 3-pyridyl)-1- methylpropyl]-2-(5- trifluoromethyl-2- pyridyloxy)-2- methylpropanamide | 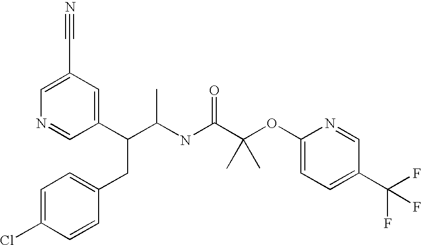 | Enan- tiomer A |
| I-50 | N-[3-(4-Chloro- phenyl)-2-(5-cyano- 3-pyridyl)-1- methylpropyl]-2-(5- trifluoromethyl-2- pyridyloxy)-2- methylpropanamide | 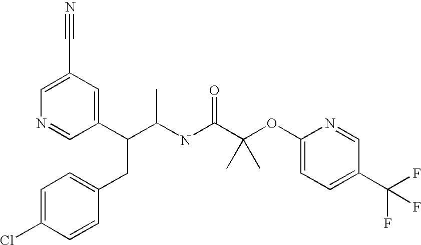 | Enan- tiomer B |
| I-51 | N-[3-(4-Chloro- phenyl)-2-(5- methyl-3-pyridyl)- 1-methylpropyl]-2- (5-trifluoromethyl- 2-pyridyloxy)-2- methylpropanamide | 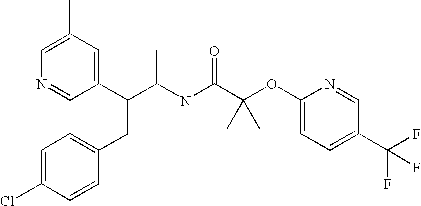 | Enan- tiomer A |
| I-52 | N-[3-(4-Chloro- phenyl)-2-(5- methyl-3-pyridyl)- 1-methylpropyl]-2- (5-trifluoromethyl- 2-pyridyloxy)-2- methylpropanamide | 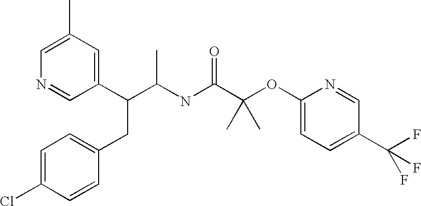 | Enan- tiomer B |
| I-53 | N-(3-(4-chloro- phenyl)-2-(7- azaindol-N-yl)-1- methyl)propyl-2-(5- trifluoromethyl-2- oxypyridine-2-yl)- 2-methyl- propanamide | 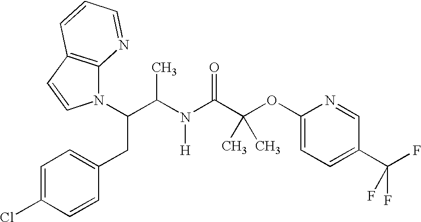 | Diastere omer B |
| I-54 | N-(3-(4-chloro- phenyl)-2-(N- methyl-N-phenyl)- amino-1-methyl)- propyl-2-(5-tri- fluoromethyl-2-oxy- pyridine-2-yl)-2- methylpropanamide | 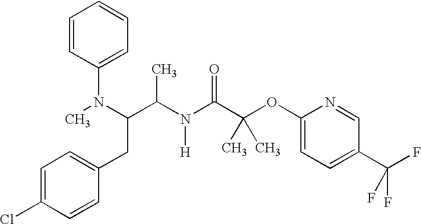 | Single enantio mer derived from Isomer B |
| I-55 | N-(3-(4-chloro- phenyl)-2-(indol-N- yl)-1-methyl)- propyl-2-(5- trifluoromethyl-2- oxypyridine-2-yl)- 2-methylpropan- amide | 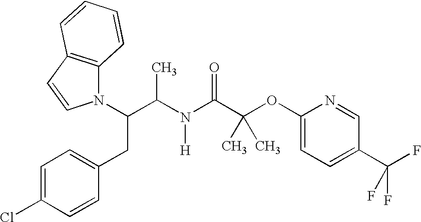 | Single enantio mer derived from Isomer B |
| I-56 | N-(3-(4-chloro- phenyl)-2-(indolin- N-yl)-1-methyl)- propyl-2-(5- trifluoromethyl-2- oxypyridine-2-yl)- 2-methyl- propanamide | 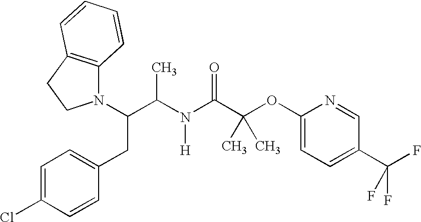 | Diastere omer B |
| I-57A | 2-Methyl-N-[1- methyl-3-(4-meth- ylphenyl)-2- phenylpropyl]-2- {[5-(trifluoro- methyl)-pyridin-2- yl]oxy}propan- amide | 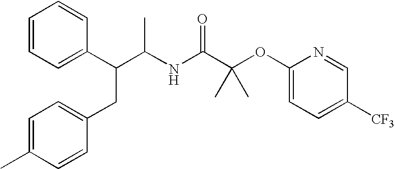 | less polar diastere omer eluted at 4.73 minutes |
| I-57B | | 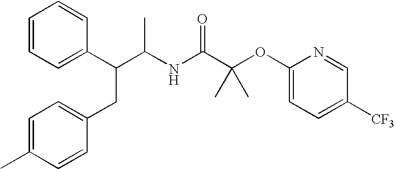 | more polar diastero mer eluted at 5.87 minutes |
| I-58 | | 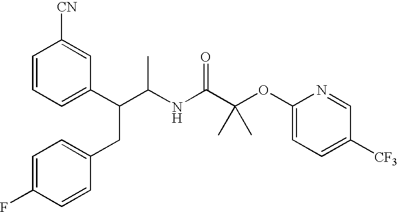 | less polar diastere omer eluted at 11.75 minutes |
| I-59 | N-(3-(4-chloro- phenyl)-1-methyl-2- (thiophen-3- yl)propyl)-2- methyl-2-(5- chloropyridin-2- yl)oxy)-2-methyl- propanamide | 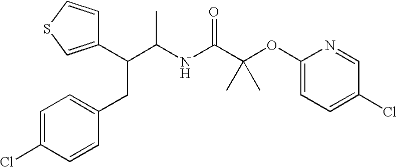 | |
| I-60 | N-(2-(3-cyano- phenyl)-1,4- dimethylpentyl)-2- methyl-2((5- (trifluoromethyl)- pyridin-2-yl)oxy)- propanamide | 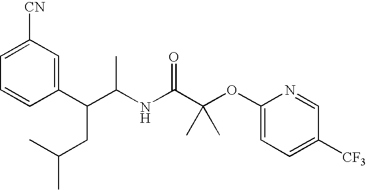 | Enan- tiomer B |
| I-61 | N-(2-(3-cyano- phenyl)-3- cyclobutyl-1- methylpropyl)-2- methyl-2((5- (trifluoromethyl)- pyridin-2-yl)oxy)- propanamide | 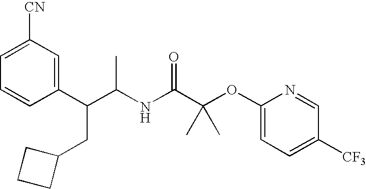 | Enan- tiomer B |
| I-62 | N-(2-(3-cyano- phenyl)-3- cyclopentyl-1- methylpropyl)-2- methyl-2((5- (trifluoromethyl)- pyridin-2-yl)oxy)- propanamide | 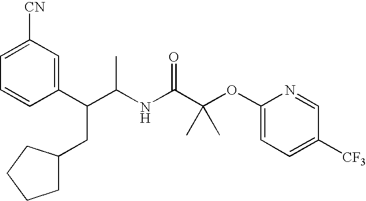 | Enan- tiomer B |
| I-63 | N-(2-(3- cyanophenyl)-3- cyclohexyl-1- methylpropyl)-2- methyl-2((5- (trifluoromethyl) pyridin-2- yl)oxy)- propanamide | 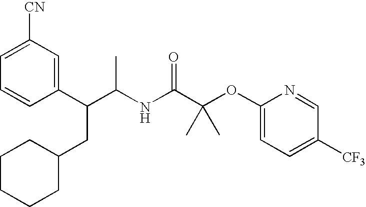 | Enan- tiomer B |
| I-64 | | 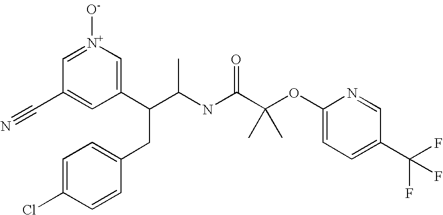 | |
| II | | 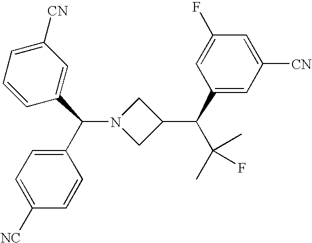 | |

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### Example 1

### Cannabinoid Receptor-1 (CB1) Binding (IC50) Assay

Binding affinity determination is based on recombinant human CB1 receptor expressed in Chinese Hamster Ovary (CHO) cells (Felder et al, Mol. Pharmacol. 48: 443-450, 1995). Total assay volume is 250 μl (240 μl CB1 receptor membrane solution plus 5 μl test compound solution plus 5 μl [3H]CP-55940 solution). Final concentration of [3H]CP-55940 is 0.6 nM. Binding buffer contains 50 mM Tris-HCl, pH7.4, 2.5 mM EDTA, 5 mM MgCl₂, 0.5 mg/mL fatty acid free bovine serum albumin and protease inhibitors (Cat#P8340, from Sigma). To initiate the binding reaction, 5 μl of radioligand solution is added, the mixture is incubated with gentle shaking on a shaker for 1.5 h at 30° C. The binding is terminated by using 96-well harvester and filtering through GF/C filter presoaked in 0.05% polyethylenimine. The bound radiolabel is quantitated using scintillation counter. Apparent binding affinities for various compounds are calculated from IC50 values (DeBlasi et al., Trends Pharmacol Sci 10: 227-229, 1989).

The compounds of Examples I-2 to I-64 and II were tested and found to have IC50 values of less than 10 micromolar. The IC 50 values for several of the compounds of formula I and for the compound of formula II are provided in the table below.

The binding assay for CB2 receptor is done similarly with recombinant human CB2 receptor expressed in CHO cells.

| Example No. | IC50 (nM) | EC50 (nM) |
|---|---|---|
| I-6 | 2.30 | 2.1 |
| I-25 | 0.45 | 1.33 |
| I-35 | 0.40 | 5.0 |
| I-36 | 0.66 | 1.25 |
| I-39 | 3.0 | 1.5 |
| I-44 | 0.29 | 2.39 |
| I-59 | 7.28 | 14.0 |
| I-60 | 1.39 | 2.0 |
| I-62 | 0.33 | 2.0 |
| I-64 | 29.41 | 13.0 |
| II | 3 | 2 |

### Example 2

### Cannabinoid Receptor-1 (CB1) Functional Activity (EC50) Assay

The functional activation of CB1 receptor is based on recombinant human CB1 receptor expressed in CHO cells (Felder et al, Mol. Pharmacol. 48: 443-450, 1995). To determine the agonist activity or inverse agonist activity of any test compound, 50 ul of CB1-CHO cell suspension are mixed with test compound and 70 ul assay buffer containing 0.34 mM 3-isobutyl-1-methylxanthine and 5.1 uM of forskolin in 96-well plates. The assay buffer is comprised of Earle's Balanced Salt Solution supplemented with 5 mM MgCl₂, 1 mM glutamine, 10 mM HEPES, and 1 mg/mL bovine serum albumin. The mixture is incubated at room temperature for 30 minutes, and terminated by adding 30 uL/well of 0.5M HCl. The total intracellular cAMP level is quantitated using the New England Nuclear Flashplate and cAMP radioimmunoassay kit.

To determine the antagonist activity of test compound, the reaction mixture also contains 0.5 nM of the agonist CP55940, and the reversal of the CP55940 effect is quantitated. Alternatively, a series of dose response curves for CP55940 is performed with increasing concentration of the test compound in each of the dose response curves. The EC 50 values for several of the compounds of formula I and for the compound of formula II are provided in the table above.

The functional assay for the CB2 receptor is done similarly with recombinant human CB2 receptor expressed in CHO cells.

### Example 3

### In Vivo Study of a CB-1 Inverse Agonist/Antagonist for the Treatment of Alzheimer's Disease

### Improvement of Spatial Recognition Memory Performance by a CB1 Antagonist in Scopolamine-Treated Mice

Materials and Methods: Male C57BL/6 mice (Taconic Farms, 2-3 months old at the beginning of the experiment) are used. Upon arrival to the facility, mice are singly housed, have free access to food and water, and are given a 1-week acclimation period prior to testing. They are kept in an animal room which is maintained at 21±2° C. temperature, 55±15% relative humidity and on a 12-hr light-dark cycle (7:00-19:00).

The spatial recognition test is used to assess recognition memory performance; it is based on the spontaneous tendency of rodents to explore an object in a novel location more often than an object in a familiar location. On the first two days of testing, mice are submitted to a daily 15-minute familiarization session to an empty open field made of Plexiglas (50×50×40 cm). On the following day, mice are submitted to a 15-minute sample trial during which they are allowed to explore the open field in the presence of two identical objects located on the same side of the arena, but in different corners. After a 24-hour delay, mice are submitted to a 15-minute test trial during which they are allowed to explore the open field in the presence of the two objects previously explored, one object remaining in the previous location (non-displaced object) and the other object placed in a novel location (displaced object). A spatial recognition index, calculated for each mouse, is expressed as the ratio (D*100)/(ND+D), where D and ND are the time spent during the test trial on the displaced object and the non-displaced object, respectively. The time spent exploring each object (nose pointing towards an object at a distance <2 cm) is recorded using a computer-assisted video tracking system (Topscan software, CleverSys Inc.).

Five treatment groups (n=12 per group) are tested in parallel: 1) vehicle-vehicle, 2) vehicle-scopolamine, 3) a CB1 antagonist/inverse antagonist/inverse agonist of formula I, such as N-[3-(4-chlorophenyl)-2-(3-cyanophenyl)-1-methylpropyl]-2-(5-trifluoromethyl-2-pyridyloxy)-2-methylpropanamide, and scopolamine (3 doses of CB1 antagonist). Scopolamine-Hydrobromide (0.3 mg/kg) or vehicle is administered subcutaneously 20 minutes prior to the sample trial. The CB1 antagonist/inverse agonist (0.3-3.0 mg/kg) or vehicle is administered intraperitoneally 30 minutes prior to the sample trial. Both drugs are administered in a volume of 10 ml/kg.

Results: Effective CB-1 inverse agonist/antagonists result in a spatial recognition index in CB1 antagonist/inverse agonist treated animals significantly above that of scopolamine-treated animals.

### Example 4

### In Vivo Study with a CB-1 Inverse Agonist/Antagonist and an Anti-Alzheimer's Disease Agent for the Treatment of Alzheimer's Disease

Materials and Methods: The study of EXAMPLE 3 is run with the following additional treatment groups with an additional treatment group (n=12): a CB1 antagonist/inverse agonist of formula I, such as N-[3-(4-chlorophenyl)-2-(3-cyanophenyl)-1-methylpropyl]-2-(5-trifluoromethyl-2-pyridyloxy)-2-methylpropanamide, and an anti-Alzheimer's disease agent, such a Donepezil hydrochloride, and scopolamine (3 doses of CB1 antagonist).

Results: Effective anti-Alzheimer's combinations result in a greater spatial recognition index when the CB-1 inverse agonist/antagonist and the anti-Alzheimer's Disease agent are given together, than the spatial recognition index seen with either compound administered alone.

### Example 5

### Human Study

### Treating Alzheimer's Disease by Improving Cognition and Function (Memory and Performing Everyday Tasks), or Preventing or Delaying the Onset of Alzheimer's Disease with a CB-1 Inverse Agonist/Antagonist in a Subject Prior to the Exhibition of Cognitive Impairment, or in a Subject Exhibiting Mild Cognitive Impairment, or in a Subject with Alzheimer's Disease

Methods: The study consists of 800 people with Alzheimer's Disease, or people exhibiting mild cognitive impairment, or people not yet exhibiting cognitive impairment as identified using the MMSE or similar diagnostic tool. After a two-week placebo (sugar pill) run-in period, the patients are randomized into 2 treatment groups: placebo; and an effective dose of a CB-1 inverse agonist/antagonist of formula I, such as 0.5 to 2.5 mg of N-[3-(4-chlorophenyl)-2-(3-cyanophenyl)-1-methylpropyl]-2-(5-trifluoromethyl-2-pyridyloxy)-2-methylpropanamide. The CB-1 inverse agonist/antagonist of formula I is given in tablet or liquid filled capsule form once or more per day, as previously determined to be effective. The cognitive status of the subject is monitored periodically over 6 months using the MMSE or similar tools/tests, and the subject is monitored for clinical symptoms of dementia. The subjects take tests, such as ADAS-cog, CANTAB, CIBIC-plus, SIB or ADCS-ADL-severe, to measure thinking, memory, and the ability to function in daily life, including effects on behavior.

Results: Compounds of formula I effective for the treatment of Alzheimer's Disease result in:
Effective improvement on the ADAS-cog test battery or the CANTAB test battery, which measure how well they think, remember, communicate, and figure things out relative to the placebo group; or
Clinical improvement in the CIBIC-plus test, an interview that measures a person's ability to function, relative to the placebo group; or
Improvement in thinking, memory, and how they functioned in daily life in the SIB test relative to the placebo group; or
Significantly less decline on the ADCS-ADL-severe test than patients in the placebo group.

### Example 6

### Human Study

### Treating Alzheimer's Disease by Improving Cognition and Function (Memory and Performing Everyday Tasks), or Preventing or Delaying the Onset of Alzheimer's Disease, with a Combination of a CB-1 Inverse Agonist/Antagonist and an Anti-Alzheimer's Disease Agent in a Subject Prior to the Exhibition of Cognitive Impairment, or in a Subject Exhibiting Mild Cognitive Impairment, or in a Subject with Alzheimer's Disease

Materials and Methods: The study consists of 800 people with Alzheimer's Disease, or people exhibiting mild cognitive impairment, or people not yet exhibiting cognitive impairment as identified using the MMSE or similar diagnostic tool. After a two-week placebo (sugar pill) run-in period, the patients are randomized into 4 treatment groups: placebo; an effective dose of a CB-1 inverse agonist/antagonist of formula I, such as 0.5 to 2.5 mg of N-[3-(4-chlorophenyl)-2-(3-cyanophenyl)-1-methylpropyl]-2-(5-trifluoromethyl-2-pyridyloxy)-2-methylpropanamide; an effective dose of an anti-Alzheimer's Disease agent, such as 5 or 10 mg of Donepezil hydrochloride; and an effective dose of the CB-1 inverse agonist/antagonist plus an effective dose of the anti-Alzheimer's Disease agent. The CB-1 inverse agonist/antagonist of formula I is given in tablet or liquid filled capsule form once or more per day, as previously determined to be effective. The anti-Alzheimer's disease agent is given in tablet form once or more per day, as previously determined to be effective. When the anti-Alzheimer's Disease agent is Donepezil hydrochloride, tablets of 5 or 10 mg are given once per day. The cognitive status of the subject is monitored periodically over 6 months using the MMSE or similar tools/tests, and the subject is monitored for clinical symptoms of dementia. The subjects take tests, such as ADAS-cog, CANTAB test battery, CIBIC-plus, SIB or ADCS-ADL-severe, to measure thinking, memory, and the ability to function in daily life, including effects on behavior.

Results: Effective anti-Alzheimer's combinations result in greater improvements or less cognitive decline in the following tests when the CB-1 inverse agonist and the anti-Alzheimer's Disease agent are given together, than the improvements or cognitive decline seen in the tests when either compound is administered alone:
Effective improvement on the ADAS-cog test battery or the CANTAB test battery, which measure how well they think, remember, communicate, and figure things out; or
Clinical improvement in the CIBIC-plus test, an interview that measures a person's ability to function; or
Improvement in thinking, memory, and how they functioned in daily life in the SIB test; or
Significantly less decline on the ADCS-ADL-severe test.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method of treating Alzheimer's disease comprising administration to a patient in need of such treatment a therapeutically effective amount of a compound of formula I: 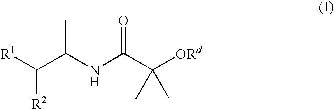
or a pharmaceutically acceptable salt thereof, wherein:
R¹ is selected from:
(1) cycloheteroalkyl,
(2) aryl,
(3) heteroaryl, and
(4) —NR^{a}R^{c},
wherein aryl and heteroaryl are optionally substituted with one to three substituents independently selected from R^{b};
R² is selected from:
(1) C₁₋₁₀alkyl,
(2) C₃₋₁₀cycloalkyl-C₁₋₄alkyl,
(3) aryl-C₁₋₄alkyl, and
(4) heteroaryl-C₁₋₄alkyl,
wherein each cycloalkyl, aryl and heteroaryl is optionally substituted with one to three substituents independently selected from R^{b};
each R^{a} is independently selected from:
(1) hydrogen,
(2) methyl, and
(3) —CF₃;
each R^{b} is independently selected from:
(1) halogen,
(2) cyano,
(3) trifluoromethyl,
(4) trifluoromethoxy,
(5) C₁₋₃alkyloxy, and
(6) C₁₋₃alkyl;
R^{c} is independently selected from:
(1) hydrogen,
(2) C₁₋₆alkyl,
(3) aryl,
(4) heteroaryl,
(5) aryl-methyl, and
(6) heteroaryl-methyl,
wherein each R^{c} may be unsubstituted or substituted with one to three substituents selected from R^{h};
R^{d} is independently selected from:
(1) cycloalkyl,
(2) aryl, and
(3) heteroaryl,
wherein each R^{d} may be unsubstituted or substituted with one to three substituents selected from R^{h};
each R^{h} is independently selected from:
(1) halogen,
(2) C₁₋₃alkyl,
(3) —CN, and
(4) —CF₃;
wherein when pyridyl groups are unsubstituted on nitrogen, they may optionally be present as the N-oxide.

2. The method according to claim 1, wherein R¹ is selected from:
(1) phenyl,
(2) pyridyl,
(3) indolyl,
(4) 7-aza-indolyl,
(5) thiophenyl, and
(6) 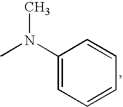
wherein each aryl and heteroaryl is optionally substituted with one or two substitutents independently selected from R^{b}, and each pyridyl may be optionally present as the N-oxide; and pharmaceutically acceptable salts thereof.

3. The method according to claim 2, wherein R¹ is selected from:
(1) phenyl,
(2) 3-cyanophenyl,
(3) 3-methylphenyl,
(4) 3,5-difluorophenyl,
(5) 3-pyridyl,
(6) 5-chloro-3-pyridyl,
(7) 5-methyl-3-pyridyl,
(8) 5-cyano-3-pyridyl,
(9) 1-oxido-5-cyano-3-pyridyl,
(10) 1-indolyl,
(11) 7-aza-indol-N-yl,
(12) 2-thiophenyl, and
(13) 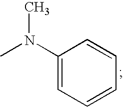
and pharmaceutically acceptable salts thereof.

4. The method according to claim 3, wherein R¹ is 5-cyano-3-pyridyl; and pharmaceutically acceptable salts thereof.

5. The method according to claim 2, wherein R² is selected from:
(1) C₁₋₆alkyl,
(2) C₃₋₆cycloalkylmethyl,
(3) phenylmethyl, and
(4) heteroarylmethyl,
wherein each cycloalkyl, aryl and heteroaryl is optionally substituted with one to three substituents independently selected from R^{b}; and pharmaceutically acceptable salts thereof.

6. The method according to claim 5, wherein R² is selected from:
(1) 2-methylpropyl,
(2) n-pentyl,
(3) cyclobutylmethyl,
(4) cyclopentylmethyl,
(5) cyclohexylmethyl,
(6) benzyl,
(7) 4-chlorobenzyl,
(8) 4-methylbenzyl,
(9) 4-fluorobenzyl,
(10) 4-methoxybenzyl, and
(11) (5-chloro-2-pyridyl)methyl;
and pharmaceutically acceptable salts thereof.

7. The method according to claim 2, wherein R^{d} is selected from:
(1) C₄₋₆cycloalkyl,
(2) aryl, and
(3) heteroaryl,
wherein R^{d} may be unsubstituted or substituted with one or two substituents selected from R^{h}; and
pharmaceutically acceptable salts thereof.

8. The method according to claim 7, wherein R^{d} is selected from:
(1) phenyl,
(2) pyridyl, and
(3) pyrimidinyl,
wherein R^{d} may be unsubstituted or substituted with one or two substituents selected from R^{h}; and
pharmaceutically acceptable salts thereof.

9. The method according to claim 8, wherein R^{d} is selected from:
(1) phenyl,
(2) 4-chlorophenyl,
(3) 3-chlorophenyl,
(4) 3,5-difluorophenyl,
(5) 3,5-dichlorophenyl,
(6) 2-pyridyl,
(7) 5-chloro-2-pyridyl,
(8) 6-methyl-2-pyridyl,
(9) 5-trifluoromethyl-2-pyridyl,
(10) 4-trifluoromethyl-2-pyridyl,
(11) 4-trifluoromethyl-2-pyrimidyl, and
(12) 6-trifluoromethyl-4-pyrimidyl;
and pharmaceutically acceptable salts thereof.

10. The method according to claim 1 wherein the compound of formula I is selected from:
(1) N-[3-(4-chlorophenyl)-1-methyl-2-phenylpropyl]-2-(4-chlorophenyloxy)-2-methylpropanamide;
(2) N-[3-(4-chlorophenyl)-1-methyl-2-phenylpropyl]-2-(2-pyridyloxy)-2-methylpropanamide;
(3) N-[3-(4-chlorophenyl)-1-methyl-2-(3-pyridyl)propyl]-2-(4-chlorophenyloxy)-2-methylpropanamide;
(4) N-[3-(4-chlorophenyl)-1-methyl-2-phenylpropyl]-2-(3,5-difluorophenyloxy)-2-methylpropanamide;
(5) N-[3-(4-chlorophenyl)-2(S)-phenyl-1(S)-methylpropyl]-2-(3,5-dichlorophenyloxy)-2-methylpropanamide;
(6) N-[3-(4-chlorophenyl)-1-methyl-2-phenylpropyl]-2-(3-chlorophenyloxy)-2-methylpropanamide;
(7) N-[3-(4-chlorophenyl)-2-(3,5-difluorophenyl)-1-methylpropyl]-2-(2-pyridyloxy)-2-methylpropanamide;
(8) N-[(2(S),3(S))-3-(4-chlorophenyl)-1-methyl-2-phenyl-propyl]-2-(5-chloro-2-pyridyloxy)-2-methylpropanamide;
(9) N-[3-(4-chlorophenyl)-1-methyl-2-phenylpropyl]-2-(6-methyl-pyridyloxy)-2-methylpropanamide;
(10) N-[3-(4-chlorophenyl)-1-methyl-2-phenylpropyl]-2-(phenyloxy)-2-methylpropanamide;
(11) N-[(3-(4-chlorophenyl)-1-methyl-2-phenylpropyl]-2-(5-trifluoromethylpyridyloxy)-2-methylpropanamide;
(12) N-[3-(4-chlorophenyl)-2-(3-pyridyl)-1-methylpropyl]-2-(5-trifluoromethyl-2-pyridyloxy)-2-methylpropanamide;
(13) N-[3-(4-chlorophenyl)-2-(3-cyanophenyl)-1-methylpropyl]-2-(5-trifluoromethyl-2-pyridyloxy)-2-methylpropanamide;
(14) N-[3-(4-chlorophenyl)-2-(5-chloro-3-pyridyl)-1-methylpropyl]-2-(5-trifluoromethyl-2-pyridyloxy)-2-methylpropanamide;
(15) N-[3-(4-chlorophenyl)-2-(5-methyl-3-pyridyl)-1-methylpropyl]-2-(5-trifluoromethyl-2-pyridyloxy)-2-methylpropanamide;
(16) N-[3-(4-chlorophenyl)-2-(5-cyano-3-pyridyl)-1-methylpropyl]-2-(5-trifluoromethyl-2-pyridyloxy)-2-methylpropanamide;
(17) N-[3-(4-chlorophenyl)-2-(3-methylphenyl)-1-methylpropyl]-2-(5-trifluoromethyl-2-pyridyloxy)-2-methylpropanamide;
(18) N-[3-(4-chlorophenyl)-2-phenyl-1-methylpropyl]-2-(4-trifluoromethyl-2-pyridyloxy)-2-methylpropanamide;
(19) N-[3-(4-chlorophenyl)-2-phenyl-1-methylpropyl]-2-(4-trifluoromethyl-2-pyrimidyloxy)-2-methylpropanamide;
(20) N-[3-(4-chlorophenyl)-1-methyl-2-(thiophen-3-yl)propyl]-2-(5-chloro-2-pyridyloxy)-2-methylpropanamide;
(21) N-[3-(5-chloro-2-pyridyl)-2-phenyl-1-methylpropyl]-2-(5-trifluoromethyl-2-pyridyloxy)-2-methylpropanamide;
(22) N-[3-(4-methyl-phenyl)-1-methyl-2-phenylpropyl]-2-(5-trifluoromethyl-phenyloxy)-2-methylpropanamide;
(23) N-[3-(4-fluoro-phenyl)-2-(3-cyano-phenyl)-1-methylpropyl]-2-(5-trifluoromethyl-2-pyridyloxy)-2-methylpropanamide;
(24) N-[3-(4-chlorophenyl)-2-(1-indolyl)-1-methyl)propyl]-2-(5-trifluoromethyl-2-oxypyridine-2-yl)-2-methylpropanamide;
(25) N-[3-(4-chlorophenyl)-2-(7-azaindol-N-yl)-1-methyl)propyl]-2-(5-trifluoromethyl-2-pyridyloxy)-2-methylpropanamide;
(26) N-[3-(4-chloro-phenyl)-2-(1-indolinyl)-1-methylpropyl]-2-(5-trifluoromethyl-2-pyridyloxy)-2-methylpropanamide;
(27) N-[3-(4-chloro-phenyl)-2-(N-methyl-anilino)-1-methylpropyl]-2-(5-trifluoromethyl-2-pyridyloxy)-2-methylpropanamide;
(28) N-[3-(4-methoxy-phenyl)-2-(3-cyano-phenyl)-1-methylpropyl]-2-(5-trifluoromethyl-2-pyridyloxy)-2-methylpropanamide;
(29) N-[3-(4-chlorophenyl)-2-(3-cyanophenyl)-1-methylpropyl]-2-(6-trifluoromethyl-4-pyrimidyloxy)-2-methylpropanamide;
(30) N-[2-(3-cyanophenyl)-1,4-dimethylpentyl]-2-(5-trifluoromethyl-2-pyridyloxy)-2-methylpropanamide;
(31) N-[3-(4-chlorophenyl)-2-(1-oxido-5-cyano-3-pyridyl]-1-methylpropyl]-2-(5-trifluoromethyl-2-pyridyloxy)-2-methylpropanamide;
(32) N-[2-(3-cyanophenyl)-3-cyclobutyl-1-methylpropyl]-2-(5-trifluoromethyl-2-pyridyloxy)-2-methylpropanamide;
(33) N-[2-(3-cyanophenyl)-1-methyl-heptyl]-2-(5-trifluoromethyl-2-pyridyloxy)-2-methylpropanamide;
(34) N-[2-(3-cyanophenyl)-3-cyclopentyl-1-methylpropyl]-2-(5-trifluoromethyl-2-pyridyloxy)-2-methylpropanamide; and
(35) N-[2-(3-cyanophenyl)-3-cyclohexyl-1-methylpropyl]-2-(5-trifluoromethyl-2-pyridyloxy)-2-methylpropanamide;
and pharmaceutically acceptable salts thereof.

11. The method according to claim 9, wherein R^{d} is 5-trifluoromethyl-2-pyridyl; and pharmaceutically acceptable salts thereof.

12. The method according to claim 11, wherein the compound of formula I is selected from:
(1) N-[(3-(4-chlorophenyl)-1-methyl-2-phenylpropyl]-2-(5-trifluoromethylpyridyloxy)-2-methylpropanamide;
(2) N-[3-(4-chlorophenyl)-2-(3-pyridyl)-1-methylpropyl]-2-(5-trifluoromethyl-2-pyridyloxy)-2-methylpropanamide;
(3) N-[3-(4-chlorophenyl)-2-(3-cyanophenyl)-1-methylpropyl]-2-(5-trifluoromethyl-2-pyridyloxy)-2-methylpropanamide;
(4) N-[3-(4-chlorophenyl)-2-(5-chloro-3-pyridyl)-1-methylpropyl]-2-(5-trifluoromethyl-2-pyridyloxy)-2-methylpropanamide;
(5) N-[3-(4-chlorophenyl)-2-(5-methyl-3-pyridyl)-1-methylpropyl]-2-(5-trifluoromethyl-2-pyridyloxy)-2-methylpropanamide;
(6) N-[3-(4-chlorophenyl)-2-(5-cyano-3-pyridyl)-1-methylpropyl]-2-(5-trifluoromethyl-2-pyridyloxy)-2-methylpropanamide;
(7) N-[3-(4-chlorophenyl)-2-(3-methylphenyl)-1-methylpropyl]-2-(5-trifluoromethyl-2-pyridyloxy)-2-methylpropanamide;
(8) N-[3-(5-chloro-2-pyridyl)-2-phenyl-1-methylpropyl]-2-(5-trifluoromethyl-2-pyridyloxy)-2-methylpropanamide;
(9) N-[3-(4-fluoro-phenyl)-2-(3-cyano-phenyl)-1-methylpropyl]-2-(5-trifluoromethyl-2-pyridyloxy)-2-methylpropanamide;
(10) N-[3-(4-chlorophenyl)-2-(1-indolyl)-1-methyl)propyl]-2-(5-trifluoromethyl-2-oxypyridine-2-yl)-2-methylpropanamide;
(11) N-[3-(4-chlorophenyl)-2-(7-azaindol-N-yl)-1-methyl)propyl]-2-(5-trifluoromethyl-2-pyridyloxy)-2-methylpropanamide;
(12) N-[3-(4-chloro-phenyl)-2-(1-indolinyl)-1-methylpropyl]-2-(5-trifluoromethyl-2-pyridyloxy)-2-methylpropanamide;
(13) N-[3-(4-chloro-phenyl)-2-(N-methyl-anilino)-1-methylpropyl]-2-(5-trifluoromethyl-2-pyridyloxy)-2-methylpropanamide;
(14) N-[3-(4-methoxy-phenyl)-2-(3-cyano-phenyl)-1-methylpropyl]-2-(5-trifluoromethyl-2-pyridyloxy)-2-methylpropanamide;
(15) N-[2-(3-cyanophenyl)-1,4-dimethylpentyl]-2-(5-trifluoromethyl-2-pyridyloxy)-2-methylpropanamide;
(16) N-[3-(4-chlorophenyl)-2-(1-oxido-5-cyano-3-pyridyl]-1-methylpropyl]-2-(5-trifluoromethyl-2-pyridyloxy)-2-methylpropanamide;
(17) N-[2-(3-cyanophenyl)-3-cyclobutyl-1-methylpropyl]-2-(5-trifluoromethyl-2-pyridyloxy)-2-methylpropanamide;
(18) N-[2-(3-cyanophenyl)-1-methyl-heptyl]-2-(5-trifluoromethyl-2-pyridyloxy)-2-methylpropanamide;
(19) N-[2-(3-cyanophenyl)-3-cyclopentyl-1-methylpropyl]-2-(5-trifluoromethyl-2-pyridyloxy)-2-methylpropanamide; and
(20) N-[2-(3-cyanophenyl)-3-cyclohexyl-1-methylpropyl]-2-(5-trifluoromethyl-2-pyridyloxy)-2-methylpropanamide;
and pharmaceutically acceptable salts thereof.

13. A method of treating an Alzheimer's disease related disorder comprising administration to a patient in need of such treatment a therapeutically effective amount of a compound of formula I: 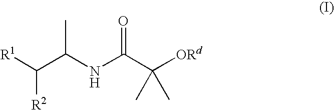
or a pharmaceutically acceptable salt thereof, wherein:
R¹ is selected from:
(1) cycloheteroalkyl,
(2) aryl,
(3) heteroaryl, and
(4) —NR^{a}R^{c},
wherein aryl and heteroaryl are optionally substituted with one to three substituents independently selected from R^{b};
R² is selected from:
(1) C₁₋₁₀alkyl,
(2) C₃₋₁₀cycloalkyl-C₁₋₄alkyl,
(3) aryl-C₁₋₄alkyl, and
(4) heteroaryl-C₁₋₄alkyl,
wherein each cycloalkyl, aryl and heteroaryl is optionally substituted with one to three substituents independently selected from R^{b};
each R^{a} is independently selected from:
(1) hydrogen,
(2) methyl, and
(3) —CF₃;
each R^{b} is independently selected from:
(1) halogen,
(2) cyano,
(3) trifluoromethyl,
(4) trifluoromethoxy,
(5) C₁₋₃alkyloxy, and
(6) C₁₋₃alkyl;
R^{c} is independently selected from:
(1) hydrogen,
(2) C₁₋₆alkyl,
(3) aryl,
(4) heteroaryl,
(5) aryl-methyl, and
(6) heteroaryl-methyl,
wherein each R^{c} may be unsubstituted or substituted with one to three substituents selected from R^{h};
R^{d} is independently selected from:
(1) cycloalkyl,
(2) aryl, and
(3) heteroaryl,
wherein each R^{d} may be unsubstituted or substituted with one to three substituents selected from R^{h};
each R^{h} is independently selected from:
(1) halogen,
(2) C₁₋₃alkyl,
(3) —CN, and
(4) —CF₃;
wherein when pyridyl groups are unsubstituted on nitrogen, they may optionally be present as the N-oxide.

14. The method according to claim 13 wherein the Alzheimer's Disease related disorder is selected from: dementia, age related cognitive decline, and mild cognitive impairment.

15-17. (canceled)

18. A pharmaceutical composition comprising
(a) an anti-Alzheimer's disease agent selected from the group consisting of:
(1) memantine,
(2) tacrine,
(3) donepezil,
(4) rivastigmine, and
(5) galantamine;
or a pharmaceutically acceptable salt thereof; and
(b) a cannabinoid antagonist/inverse agonist of formula I 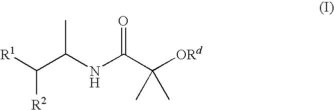
or a pharmaceutically acceptable salt thereof, wherein:
R¹ is selected from:
(1) cycloheteroalkyl,
(2) aryl,
(3) heteroaryl, and
(4) —NR^{a}R^{c},
wherein aryl and heteroaryl are optionally substituted with one to three substituents independently selected from R^{b};
R² is selected from:
(1) C₁₋₁₀alkyl,
(2) C₃₋₁₀cycloalkyl-C₁₋₄alkyl,
(3) aryl-C₁₋₄alkyl, and
(4) heteroaryl-C₁₋₄alkyl,
wherein each cycloalkyl, aryl and heteroaryl is optionally substituted with one to three substituents independently selected from R^{b};
each R^{a} is independently selected from:
(1) hydrogen,
(2) methyl, and
(3) —CF₃;
each R^{b} is independently selected from:
(1) halogen,
(2) cyano,
(3) trifluoromethyl,
(4) trifluoromethoxy,
(5) C₁₋₃alkyloxy, and
(6) C₁₋₃alkyl;
R^{c} is independently selected from:
(1) hydrogen,
(2) C₁₋₆alkyl,
(3) aryl,
(4) heteroaryl,
(5) aryl-methyl, and
(6) heteroaryl-methyl,
wherein each R^{c} may be unsubstituted or substituted with one to three substituents selected from R^{h};
R^{d} is independently selected from:
(1) cycloalkyl,
(2) aryl, and
(3) heteroaryl,
wherein each R^{d} may be unsubstituted or substituted with one to three substituents selected from R^{h};
each R^{h} is independently selected from:
(1) halogen,
(2) C₁₋₃ alkyl,
(3) —CN, and
(4) —CF₃;
wherein when pyridyl groups are unsubstituted on nitrogen, they may optionally be present as the N-oxide;
and pharmaceutically acceptable salts and esters thereof.

19. A method of treating Alzheimer's disease or an Alzheimer's Disease related disorder comprising administration to a patient in need of such treatment a therapeutically effective amount of the composition of claim 18.

20. A method of treating Alzheimer's disease or an Alzheimer's Disease related disorder comprising administration to a patient in need of such treatment a therapeutically effective amount of the compound of formula I-35 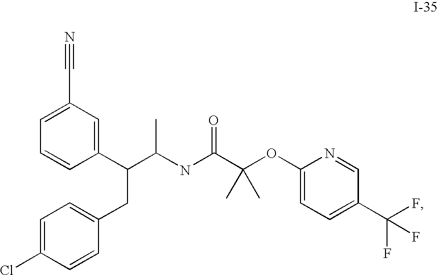
or a pharmaceutically acceptable salt thereof.

21. A method of treating Alzheimer's disease or an Alzheimer's Disease related disorder comprising administration to a patient in need of such treatment a therapeutically effective amount of the compound of formula II 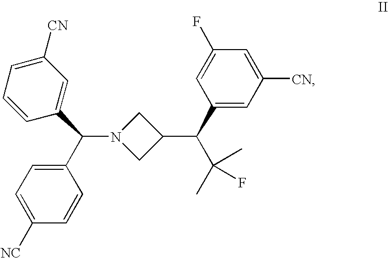
or a pharmaceutically acceptable salt thereof.

22-25. (canceled)
